(19) United States
(12) Patent Application Publication
(10) Pub. No.: US 2020/0188097 A1
(43) Pub. Date: Jun. 18, 2020

(54) Compressible Bileaflet Frame for Side Delivered Transcatheter Heart Valve

(71) Applicant: VDYNE, LLC, Maple Grove, MN (US)

(72) Inventors: CHAD PERRIN, Andover, MN (US); NEELAKANTAN SAIKRISHNAN, Plymouth, MN (US); MARK CHRISTIANSON, Plymouth, MN (US); ROBERT VIDLUND, FOREST LAKE, MN (US)

(21) Appl. No.: 16/711,415

(22) Filed: Dec. 11, 2019

Related U.S. Application Data
(60) Provisional application No. 62/778,769, filed on Dec. 12, 2018.

(51) Int. Cl. A61F 2/24

(52) U.S. Cl. CPC A61F 2/2418; A61F 2/2409; A61F 2230/0008; A61F 2210/0014; A61F 2/2433

(57) ABSTRACT

The invention relates to a compressible bileaflet frame as a flow control component for a side delivered transcatheter prosthetic valve, and in particular a side delivered transcatheter prosthetic valve wherein the valve and leaflet frame are compressible to a compressed configuration for sideways or lateral introduction (orthogonal) into the body using a delivery catheter for implanting at a desired location in the body, where the compressed configuration has a long-axis oriented roughly perpendicular to the central axis of the native annulus, wherein the long-axis of the compressed configuration of the valve is substantially parallel to a length-wise cylindrical axis of the delivery catheter, and wherein the valve has a height of about 5-60 mm and a diameter of about 25-80 mm.

### INVENTORSHIP

Provided by Application Data Sheet per USPTO rules.

### CROSS-REFERENCE TO RELATED APPLICATIONS

Provided by Application Data Sheet per USPTO rules.

### STATEMENT REGARDING FEDERALLY SPONSORED R&D

Provided by Application Data Sheet per with USPTO rules.

### NAMES OF PARTIES TO JOINT RESEARCH AGREEMENT

Provided by Application Data Sheet per with USPTO rules.

### REFERENCE TO SEQUENCE LISTING

Provided by Application Data Sheet per USPTO rules.

### STATEMENT RE PRIOR DISCLOSURES

Provided by Application Data Sheet per USPTO rules.

### BACKGROUND OF THE INVENTION

### Field of the Invention

The invention relates to a Compressible Bileaflet Frame for a Side Delivered Transcatheter Heart Valve.

### Description of the Related Art

In 1952 surgeons implanted the first mechanical heart valve. This first valve was a ball valve and it was designed by Dr. Charles Hufnagel. The recipient of this valve was a 30-year-old woman who could lead a normal life after the surgery. However, one downside of this design was that it could only be placed in the descending aorta instead of the heart itself. For this reason it did not fully correct the valve problem, only alleviate the symptoms. However it was a significant achievement because it proved that synthetic materials could be used to create heart valves.

In 1960, a new type of valve was invented and was successfully implanted. This valve is the Starr-Edwards ball valve, named after its originators. This valve was a modification of Hufnagel's original valve. The ball of the valve was slightly smaller and caged from both sides so it could be inserted into the heart itself.

The next development was tilting disc technology which was introduced in the late 1960s. These valves were a great improvement over the ball designs. The tilting dic technology allowed blood to flow in a more natural way while reducing damage to blood cells from mechanical forces. However, the struts of these valves tended to fracture from fatigue over time. As of 2003, more than 100,000 Omniscience and 300,000 Hall-Kaster/Medtronic-Hall tilting disc valves were implanted with essentially no mechanical failure.

In 1977, bi-leaflet heart valves were introduced by St. Jude. Similar to a native heart valve, blood flows directly through the center of the annulus of pyrolytic carbon valves mounted within nickel-titanium housing which makes these valves superior to other designs. However, a downside of this design is that it allows some regurgitation. A vast majority of mechanical heart valves used today have this design. As of 2003, more than 1.3 million St. Jude valves were deployed and over 500,000 Carbomedics valves with no failures to leaflets or housing. It should be noted that the human heart beats about 31 million times per year.

Development continues with compressible valves that are delivered via a catheter instead of requiring the trauma and complications of open heart surgery. This means that a cardiologist trained in endoscopy can, in theory, deploy a heart valve replacement during an outpatient procedure.

However, existing transcatheter valves are delivered by radially compressing the valves into a delivery catheter and pushing the valves out of the delivery catheter for either superelastic-expansion using shape memory alloys or by balloon expansion. However, radially compression necessarily limits the size of the valve to the size permitted by the internal diameter of the delivery catheter. In practice, this has meant that large valves, e.g. 40-60 mm in diameter, have too much wire mesh frame, pericardial covering, and internal leaflet assembly, to be delivered using transcatheter techniques.

Another problem arising in the prior art is that delivering a valve sometimes requires accessing the delivery site in the heart at difficult angles, or requiring very long, complex deployment processes, or by perforating the heart to access the ventricle. Additionally, a problem with stent-style replacement valves is that they often continue to have the regurgitation or leakage problems of prior generations of valves, as well as require expensive materials engineering in order to cope with the 100's of millions of cycles encountered during just a few years of normal heart function. A further problem arises from the assembly of valve components, and specifically the inner leaflet frame, in such a manner that allows for proper compression of the valve for delivery, while maintaining optimal expansion characteristics of the valve when released from the delivery catheter and deployed in the native annulus. Accordingly, there is still a need for alternative and simpler solutions to addressing valve-related heart pathologies.

### BRIEF SUMMARY OF THE INVENTION

Accordingly, the present invention is directed to a compressible bileaflet frame for a side delivered transcatheter prosthetic valves, comprising:
an annular ring having a distal strut and a proximal strut opposite the distal strut, and
a first leaflet and a second leaflet disposed within the annular ring forming a reciprocating flow control component, each leaflet comprised of pericardium or a biocompatible material,
wherein the annular ring comprises a first semi-ellipsoidal frame member and a second semi-ellipsoidal frame member, each semi-ellipsoidal frame having a distal end and a proximal end, wherein the distal end of the first semi-ellipsoidal frame member and the second semi-ellipsoidal are adjacent, and wherein the proximal end of the first semi-ellipsoidal frame member and the second semi-ellipsoidal are adjacent,
wherein the distal strut is comprised of a first distal post attached length-wise to a second distal post, and the proximal strut is comprised of a first proximal post attached length-wise to a second proximal post, wherein the distal strut and the proximal strut are oriented in an uncompressed configuration at an angle of between 45-135 degrees to a plane of the annular ring,
wherein the first leaflet is attached along a upper portion to the first semi-ellipsoidal frame member, and the first leaflet is attached at a distal portion to the first distal post and at a proximal portion to the first proximal post,
wherein the second leaflet is attached along a upper portion to the second semi-ellipsoidal frame member, and the second leaflet is attached at a distal portion to the second distal post and at a proximal portion to the second proximal post, and
wherein the annular ring has a diameter of about 25-80 mm and the distal and proximal struts have a length of about 5-60 mm.

In another non-limiting preferred embodiment of the invention, there is provided a compressible bileaflet frame, wherein the distal and proximal struts are oriented in an uncompressed configuration at an angle of about 90 degrees to the plane of the annular ring.

In another non-limiting preferred embodiment of the invention, there is provided a compressible bileaflet frame, wherein said compressible bileaflet frame is comprised of a braided, wire, or laser-cut wire frame.

In another non-limiting preferred embodiment of the invention, there is provided a compressible bileaflet frame, wherein the annular ring, distal strut and proximal strut are a unitary construction.

In another non-limiting preferred embodiment of the invention, there is provided a compressible bileaflet frame, wherein the first semi-ellipsoidal frame member and second semi-ellipsoidal frame member form a circle, wherein a bisecting axis of each of the first semi-ellipsoidal frame member and second semi-ellipsoidal frame member is equal to a diameter of of each of the first semi-ellipsoidal frame member and a second semi-ellipsoidal frame member.

In another non-limiting preferred embodiment of the invention, there is provided a compressible bileaflet frame, wherein the first semi-ellipsoidal frame member and second semi-ellipsoidal frame member are formed from a flat band of superelastic shape-memory alloy, or from a round-wire form of a superelastic shape-memory alloy, or a composite of a flat band and a round-wire form of a superelastic shape-memory alloy.

In another non-limiting preferred embodiment of the invention, there is provided a compressible bileaflet frame, wherein the distal strut and proximal strut are formed from a flat band of superelastic shape-memory alloy.

In another non-limiting preferred embodiment of the invention, there is provided a compressible bileaflet frame, wherein the first leaflet and the second leaflet coapt along a symmetric long-axis (distal-proximal), or coapt along a symmetric short-axis (septal-anterior), or coapt along an asymmetric axis.

In another non-limiting preferred embodiment of the invention, there is provided a compressible bileaflet frame, wherein at least one of the first or second leaflets is supported with one or more longitudinal supports integrated into or mounted upon the pericardium or biocompatible material, the one or more longitudinal supports selected from rigid or semi-rigid posts, rigid or semi-rigid ribs, rigid or semi-rigid battons, rigid or semi-rigid panels, and combinations thereof.

In another non-limiting preferred embodiment of the invention, there is provided a side delivered transcatheter prosthetic heart valve, comprising:
a self-expanding annular support frame, said annular support frame having a central channel and an outer perimeter wall circumscribing a central vertical axis in an expanded configuration,
said perimeter wall having a front wall portion and a back wall portion, the front wall portion and the back wall portion connected along a proximal side to a proximal fold area, and the front wall portion and the back wall portion connected along a distal side to a distal fold area,
the front wall portion having a front upper collar portion and a front lower body portion, the back wall portion having a back upper collar portion and a back lower body portion,
a flow control component mounted within the annular support frame and configured to permit blood flow in a first direction through an inflow end of the valve and block blood flow in a second direction, opposite the first direction, through an outflow end of the valve, wherein the flow control component is a compressible bileaflet frame comprising (i) an annular ring having a distal strut and a proximal strut opposite the distal strut, and (ii) a first leaflet and a second leaflet disposed within the annular ring, each leaflet comprised of pericardium or a biocompatible material, wherein the annular ring comprises a first semi-ellipsoidal frame member and a second semi-ellipsoidal frame member, each semi-ellipsoidal frame having a distal end and a proximal end, wherein the distal end of the first semi-ellipsoidal frame member and the second semi-ellipsoidal are adjacent, and wherein the proximal end of the first semi-ellipsoidal frame member and the second semi-ellipsoidal are adjacent, wherein the distal strut is comprised of a first distal post attached length-wise to a second distal post, and the proximal strut is comprised of a first proximal post attached length-wise to a second proximal post, wherein the distal strut and the proximal strut are oriented in an uncompressed configuration at an angle of between 45-135 degrees to a plane of the annular ring, wherein the first leaflet is attached along a upper portion to the first semi-ellipsoidal frame member, and the first leaflet is attached at a distal portion to the first distal post and at a proximal portion to the first proximal post, wherein the second leaflet is attached along a upper portion to the second semi-ellipsoidal frame member, and the second leaflet is attached at a distal portion to the second distal post and at a proximal portion to the second proximal post, and
wherein the annular ring has a diameter of about 25-80 mm and the distal and proximal struts have a length of about 5-60 mm.
wherein the valve is compressible to a compressed configuration for introduction into the body using a delivery catheter for implanting at a desired location in the body, said compressed configuration is oriented along a horizontal axis at an intersecting angle of between 45-135 degrees to the central vertical axis, and expandable to an expanded configuration having a horizontal axis at an intersecting angle of between 45-135 degrees to the central vertical axis,
wherein the horizontal axis of the compressed configuration of the valve is substantially parallel to a length-wise cylindrical axis of the delivery catheter,
wherein the valve has a height of about 5-60 mm and a diameter of about 25-80 mm.

In another non-limiting preferred embodiment of the invention, there is provided a side delivered transcatheter prosthetic heart valve, wherein the annular support frame is comprised of a plurality of compressible wire cells having a orientation and cell geometry substantially orthogonal to the central vertical axis to minimize wire cell strain when the annular support frame is configured in a vertical compressed configuration, a rolled compressed configuration, or a folded compressed configuration.

In another non-limiting preferred embodiment of the invention, there is provided a side delivered transcatheter prosthetic heart valve, wherein the front lower body portion and the back lower body portion in an expanded configuration form a shape selected from a funnel, cylinder, flat cone, or circular hyperboloid.

In another non-limiting preferred embodiment of the invention, there is provided a side delivered transcatheter prosthetic heart valve, wherein said annular support frame is comprised of a braided, wire, or laser-cut wire frame, and said annular support frame is covered with a biocompatible material.

In another non-limiting preferred embodiment of the invention, there is provided a side delivered transcatheter prosthetic heart valve, wherein the annular support frame has a side profile of a flat cone shape having a diameter R of 40-80 mm, a diameter r of 20-60 mm, and a height of 5-60 mm.

In another non-limiting preferred embodiment of the invention, there is provided a side delivered transcatheter prosthetic heart valve, wherein the annular support frame has an inner surface and an outer surface, said inner surface and said outer surface covered with a biocompatible material selected from the following consisting of: the inner surface covered with pericardial tissue, the outer surface covered with a woven synthetic polyester material, and both the inner surface covered with pericardial tissue and the outer surface covered with a woven synthetic polyester material.

In another non-limiting preferred embodiment of the invention, there is provided a side delivered transcatheter prosthetic heart valve, wherein the annular support frame has a side profile of an hourglass shape having a top diameter R1 of 40-80 mm, a bottom diameter R2 of 50-70 mm, an internal diameter r of 20-60 mm, and a height of 5-60 mm.

In another non-limiting preferred embodiment of the invention, there is provided a side delivered transcatheter prosthetic heart valve, wherein the valve in an expanded configuration has a central vertical axis that is substantially parallel to the first direction.

In another non-limiting preferred embodiment of the invention, there is provided a side delivered transcatheter prosthetic heart valve, wherein the flow control component has an internal diameter of 20-60 mm and a height of 10-40 mm, and a plurality of leaflets of pericardial material joined to form a rounded cylinder at an inflow end and having a flat closable aperture at an outflow end.

In another non-limiting preferred embodiment of the invention, there is provided a side delivered transcatheter prosthetic heart valve, wherein the flow control component is supported with one or more longitudinal supports integrated into or mounted upon the flow control component, the one or more longitudinal supports selected from rigid or semi-rigid posts, rigid or semi-rigid ribs, rigid or semi-rigid battons, rigid or semi-rigid panels, and combinations thereof.

In another non-limiting preferred embodiment of the invention, there is provided a side delivered transcatheter prosthetic heart valve, comprising a tension arm extending from a distal side of the annular support frame as an RVOT tab, the tension arm comprised of wire loop or wire frame, integrated frame section, or stent, extending from about 10-40 mm away from the annular support frame.

In another non-limiting preferred embodiment of the invention, there is provided a side delivered transcatheter prosthetic heart valve, comprising (i) an upper tension arm attached to a distal upper edge of the annular support frame, the upper tension arm comprised of wire loop or wire frame extending from about 2-20 mm away from the annular support frame, and (ii) a lower tension arm as an RVOT tab extending from a distal side of the annular support frame, the lower tension arm comprised of wire loop or wire frame, integrated frame section, or stent, extending from about 10-40 mm away from the annular support frame.

In another non-limiting preferred embodiment of the invention, there is provided a side delivered transcatheter prosthetic heart valve, comprising at least one tissue anchor connected to the annular support frame for engaging native tissue.

In another non-limiting preferred embodiment of the invention, there is provided a side delivered transcatheter prosthetic heart valve, wherein the front wall portion is a first flat panel and the back wall portion is a second flat panel, and wherein the proximal fold area and the distal fold area each comprise a sewn seam, a fabric panel, or a rigid hinge.

In another non-limiting preferred embodiment of the invention, there is provided a side delivered transcatheter prosthetic heart valve, wherein the proximal fold area and the distal fold area, each comprise a flexible fabric span without any wire cells.

In another non-limiting preferred embodiment of the invention, there is provided a side delivered transcatheter prosthetic heart valve, wherein the annular support frame is comprised of compressible wire cells selected from the group consisting of braided-wire cells, laser-cut wire cells, photolithography produced wire cells, 3D printed wire cells, wire cells formed from intermittently connected single strand wires in a wave shape, a zig-zag shape, or spiral shape, and combinations thereof.

In another non-limiting preferred embodiment of the invention, there is provided a method for compressing the implantable prosthetic heart valve as claimed and disclosed herein for length-wise orthogonal or side-long release of the valve from a delivery catheter, comprising the steps:
flattening, rolling or folding the implantable prosthetic heart valve of claim 10 into a compressed configuration wherein the long-axis of the compressed configuration of the valve is substantially parallel to a length-wise cylindrical axis of the delivery catheter,
wherein the implantable prosthetic heart valve comprises an annular support frame having a flow control component mounted within the annular support frame and configured to permit blood flow in a first direction through an inflow end of the valve and block blood flow in a second direction, opposite the first direction, through an outflow end of the valve,
wherein the valve has a height of about 5-60 mm and a diameter of about 25-80 mm.

In another non-limiting preferred embodiment of the invention, there is provided a method for compressing, wherein the implantable prosthetic heart valve is rolled or folded into a compressed configuration using a step selected from the group consisting of:

(i) unilaterally rolling into a compressed configuration from one side of the annular support frame;
(ii) bilaterally rolling into a compressed configuration from two opposing sides of the annular support frame;
(iii) flattening the annular support frame into two parallel panels that are substantially parallel to the long-axis, and then rolling the flattened annular support frame into a compressed configuration; and
(iv) flattening the annular support frame along a vertical axis to reduce a vertical dimension of the valve from top to bottom.
In another non-limiting preferred embodiment of the invention, there is provided a method for side delivery of the implantable prosthetic heart valve as claimed and disclosed herein to a desired location in the body, the method comprising the steps:
advancing a delivery catheter to the desired location in the body and delivering the expandable prosthetic heart valve as claimed and disclosed herein to the desired location in the body by releasing the valve from the delivery catheter

In another non-limiting preferred embodiment of the invention, there is provided a method for side delivery, wherein releasing the valve from the delivery catheter is selected from the steps consisting of: (i) pulling the valve out of the delivery catheter using a rigid elongated pushing rod/draw wire that is releasably connected to the distal side of the valve, wherein advancing the pushing rod away from the delivery catheter pulls the compressed valve out of the delivery catheter, or (ii) pushing the valve out of the delivery catheter using a rigid elongated pushing rod that is releasably connected to the proximal side of the valve, wherein advancing the pushing rod out of from the delivery catheter pushes the compressed valve out of the delivery catheter.

In another non-limiting preferred embodiment of the invention, there is provided a method for side delivery, comprising the additional step of anchoring one or more tissue anchors attached to the valve into native tissue.

In another non-limiting preferred embodiment of the invention, there is provided a method for side delivery, comprising the additional step of positioning a tension arm of the heart valve prosthesis into the right ventricular outflow tract of the right ventricle.

In another non-limiting preferred embodiment of the invention, there is provided a method for side delivery, comprising the additional steps of positioning a lower tension arm of the heart valve prosthesis into the right ventricular outflow tract of the right ventricle, and positioning an upper tension arm into a supra-annular position, and the upper tension arm providing a supra-annular downward force in the direction of the ventricle and lower tension arm providing a sub-annular upward force in the direction of the atrium.

In another non-limiting preferred embodiment of the invention, there is provided a method for side delivery, comprising the the additional step of rotating the heart valve prosthesis using a steerable catheter along an axis parallel to the plane of the valve annulus, wherein an upper tension arm mounted on the valve is conformationally pressure locked against supra-annular tissue, and wherein a lower tension arm mounted on the valve is conformationally pressure locked against sub-annular tissue.

### BRIEF DESCRIPTION OF THE SEVERAL VIEWS OF DRAWING

FIG. 1 is an illustration of a front side (plan) view, with a cut-away view of a leaflet frame as a flow control component according to the present invention shown mounted within a side delivered transcatheter prosthetic heart valve.

FIG. 2 is an illustration of a perspective detail view from below, of an annular ring and struts of a leaflet frame in an expanded configuration of a flow control component.

FIG. 3 is an illustration of a perspective view an annular ring and struts of a leaflet frame in a flattened compressed (rollable) configuration of a flow control component according to the present invention.

FIG. 4 is an illustration of a front plan view, of an annular ring and struts of a leaflet frame in a flattened compressed (rollable) configuration of a flow control component showing struts in partial roll-up compression.

FIG. 5 is an illustration of a perspective view from below, of a leaflet frame with leaflets as a a flow control component in a side delivered transcatheter prosthetic heart valve according to the present invention having a braid or laser-cut construction for the tubular frame, with an extended tab or tension arm that extends away from the tubular frame.

FIG. 6 is an illustration of a side view of a heart valve outline with a position of the leaflet frame according to the present invention.

FIG. 7 is an illustration of an inverted perspective view, of an annular ring and struts of a leaflet frame as a flow control component in an expanded configuration.

FIG. 8 is an illustration of an inverted exploded view, of an annular ring and struts of a leaflet frame as a flow control component in an expanded configuration.

FIG. 9 is an illustration of a bottom view of a leaflet frame according to the present invention with annular ring, struts, and leaflets as a flow control component in an open leaflet configuration.

FIG. 10 is an illustration of a bottom view of a leaflet frame according to the present invention with annular ring, struts, and leaflets as a flow control component in a closed leaflet configuration.

FIG. 11 is an illustration of a front view of a leaflet frame with annular ring, struts, and leaflets as a flow control component in a closed leaflet configuration with an outline of a side-delivered valve, and showing the leaflet frame positioned in a lower aspect of the valve body with struts extending ventricularly.

FIG. 12 is an illustration of an inverted exploded view of an annular ring and struts according to the present invention of a rectilinear leaflet frame as a flow control component in an expanded configuration.

FIG. 13 is an illustration of an inverted view of an annular ring and struts of a rectilinear leaflet frame as a flow control component in an expanded configuration showing struts aligned and connected length-wise.

FIG. 14 is an illustration of a perspective view from below of a leaflet frame with leaflets as a flow control component in a side delivered transcatheter prosthetic heart valve according to the present invention having a braid or laser-cut construction for the tubular frame, with an extended tab or tension arm that extends away from the tubular frame.

FIG. 15 is an illustration of a top view of a valve according to the present invention showing position of the leaflets in relation to the outer frame.

FIG. 16 is an illustration of a top view of an ellipse configuration of bileaflets according to the present invention.

FIG. 17 is an illustration of a top view of a circular ellipse configuration of bileaflets.

FIG. 18 is an illustration of a top view of a bi-leaflet pair in a long axis symmetric configuration.

FIG. 19 is an illustration of a top view of a bi-leaflet pair in a short axis symmetric configuration.

FIG. 20 is an illustration of a top view of a bi-leaflet pair in an asymmetric configuration.

FIG. 21 is an illustration of a front perspective view of an annular ring with coaptation spacers providing improved leaflet sealing of a leaflet frame as a flow control component in an expanded uncompressed (rollable) configuration showing struts aligned length-wise.

FIG. 22 is an illustration of a front perspective view of an annular ring with flared coaptation spacers providing improved leaflet sealing of a leaflet frame as a flow control component in an expanded uncompressed (rollable) configuration showing struts aligned length-wise.

FIG. 23 is an illustration of a front perspective view of an annular ring with split-flared coaptation spacers providing improved leaflet sealing of a leaflet frame as a flow control component in an expanded uncompressed (rollable) configuration showing struts aligned length-wise.

FIG. 24 is an illustration of a front perspective view of a variation of an annular ring made from a non-planar structure, e.g. rounded structure or wire, for a leaflet frame as a flow control component in an expanded uncompressed (rollable) configuration.

FIG. 25 is an illustration of a front perspective view of a composite variation of a leaflet frame having a flat band annular ring and round wire struts as a flow control component in an expanded uncompressed (rollable) configuration.

FIG. 26 is an illustration of a front perspective view of a composite variation of a leaflet frame having a non-planar annular ring e.g. rounded, semi-round, or wire, and flat band struts as a flow control component in an expanded uncompressed (rollable) configuration.

FIG. 27 is an illustration of a front perspective view of a valve prosthesis having a bi-leaflet frame mounted within a central aperture of an atrial collar attached to a valve body portion and having an RVOT tab extending from the lower part of the valve body portion.

FIG. 28 is an illustration of a top view of a valve prosthesis having a bi-leaflet frame as a flow control component mounted within a central aperture of an atrial collar attached to a valve body portion and having an RVOT tab extending from the lower part of the valve body portion.

FIG. 29 is an illustration of a side view of a valve prosthesis having a leaflet frame with atrial collar attached to a valve body portion and showing leaflet attachment on a frame edge and leaflet free edge.

FIG. 30 is an illustration of a side view of a pair of leaflets and shows a parabolic shape with attachment edges along the top and side edges, and a free (coaptation) edge along the bottom edge.

FIG. 31 is an illustration of a top view of a pair of leaflets, one long, one short, in one embodiment according to the present invention.

FIG. 32 is an illustration of a perspective view of a pair of leaflets, one long, one short, in one embodiment according to the present invention.

FIG. 33 is an illustration of a top view of a pair of leaflets, equal in lateral length and arranged as interconnected S shapes, in one embodiment according to the present invention.

FIG. 34 is an illustration of a perspective view of a pair of leaflets, equal in lateral length and arranged as interconnected S shapes, in one embodiment according to the present invention.

FIG. 35 is an illustration of a top view of a valve prosthesis having a bi-leaflet frame as a flow control component mounted along a short axis—septal to anterior—within a central aperture of an atrial collar attached to a valve body portion and having an RVOT tab extending from the lower part of the valve body portion.

FIG. 36 is an illustration of a top perspective view of a valve prosthesis having a bi-leaflet frame as a flow control component mounted along a long axis—distal (pulmonary artery) to proximal (inferior vena cava)—within a central aperture of an atrial collar attached to a valve body portion and having an RVOT tab extending from the lower part of the valve body portion.

FIG. 37 is an illustration of a side cut-away view of the heart.

FIG. 38 is an illustration of a side-delivered valve prosthesis deployed within the native tricuspid annulus with the inferior vena cava and the pulmonary artery.

FIG. 39 is an illustration of a delivery catheter extending out from the inferior vena cava from a femoral vein access with a (height) compressed orthogonally (side) deliverable valve disposed within the lumen of the catheter and having deployment rod attached to the near/proximal side of the compressed valve for advancing the valve out of the catheter.

FIG. 40 is an illustration of a delivery catheter extending out from the inferior vena cava from a femoral vein access with a partially released partially height compressed orthogonally (side) deliverable valve being seated against the distal side of the native tricuspid annulus with a curved (concave) valve perimeter wall (body portion), distal side, catching the native annulus within the channel formed by the concave perimeter wall, with part of the still compressed valve disposed within the lumen of the catheter and having deployment rod attached to the near/proximal side of the compressed valve for advancing the valve out of the catheter. Importantly, the ability to provide a gradual transition from native blood flow to prosthetic blood flow is a critical aspect, by incrementally expelling the compressed valve to commence partial flow through the partially expelled valve, culminating in a transition to full blood flow through the prosthetic valve. This smooth transition avoids a damaging and catastrophic “dry pump” reaction of the heart called a sphincter effect.

FIG. 41 is an illustration of a fully deployed prosthetic valve seated within the native tricuspid annulus.

FIG. 42 is an illustration of a delivery catheter extending out from the inferior vena cava from a femoral vein access with a (rolled) compressed orthogonally (side) deliverable valve disposed within the lumen of the catheter and having deployment rod attached to the near/proximal side of the compressed valve for advancing the valve out of the catheter.

FIG. 43 is an illustration of a delivery catheter extending out from the inferior vena cava from a femoral vein access with a partially released partially roll-compressed orthogonally (side) deliverable valve being seated against the distal side of the native tricuspid annulus with a curved (concave) valve perimeter wall (body portion), distal side, catching the native annulus within the channel formed by the concave perimeter wall, with part of the still compressed valve disposed within the lumen of the catheter and having deployment rod attached to the near/proximal side of the compressed valve for advancing the valve out of the catheter. Importantly, the ability to provide a gradual transition from native blood flow to prosthetic blood flow is a critical aspect, by incrementally expelling the compressed valve to commence partial flow through the partially expelled valve, culminating in a transition to full blood flow through the prosthetic valve. This smooth transition avoids a damaging and catastrophic “dry pump” reaction of the heart called a sphincter effect.

FIG. 44 is an illustration of a fully deployed prosthetic valve seated within the native tricuspid annulus.

FIG. 45 is an illustration of a side view of a leaflet frame viewed against an outline of a side-delivered prosthetic valve.

FIG. 46 is an illustration of a top, length-wise view down the horizontal-axis (proximal to distal) of a leaflet frame with band or paddle struts in a narrow aspect and annular ring in a wider, flattened aspect.

FIG. 47 is an illustration of a side view of a leaflet frame with a detail zoom of a strut in a band or paddle configuration, with wide axis showing a direction of rigid strut strength and narrow axis showing a direction of flexible strut fold-ability or roll-ability.

FIG. 48 is an illustration of a top-perspective length-wise view down the horizontal-axis (proximal to distal) of a leaflet frame with band or paddle struts in a narrow aspect and annular ring in a wider, flattened aspect, showing paired, parallel band or paddle struts.

FIG. 49 is an illustration of a side view of one preferred embodiment of a criss-crossed folding direction for struts in a compressed configuration.

FIG. 50 is an illustration of a side view of one preferred embodiment of a distal parallel folding direction for struts in a compressed configuration.

FIG. 51 is an illustration of a side view of one preferred embodiment of a proximal parallel folding direction for struts in a compressed configuration.

FIG. 52 is an illustration of a side view of one preferred embodiment of an internal facing in-plane folding direction for struts in a compressed configuration.

FIG. 53 is an illustration of a side view of one preferred embodiment of an internal facing out-of-plane folding direction for struts in a compressed configuration.

FIG. 54 is an illustration of a side view a leaflet frame as viewed against an outline of a uncompressed pre-rolled (for roll-compression vs. height compression) orthogonal prosthetic valve.

FIG. 55 is an illustration of a side view a leaflet frame as viewed against an outline of a partially compressed mid-roll (for roll-compression vs. height compression) orthogonal prosthetic valve.

FIG. 56 is an illustration of a side view a leaflet frame as viewed against an outline of a fully compressed completely rolled (for roll-compression vs. height compression) orthogonal prosthetic valve.

### DETAILED DESCRIPTION OF THE INVENTION

The invention is directed to a transcatheter heart valve replacement that is a low profile, orthogonally (side) delivered implantable prosthetic valve having an ring-shaped tubular frame, an inner 2- or 3-panel sleeve, an elongated sub-annular tension arm extending into the right ventricular outflow tract, and one or more anchor elements.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Rather, these embodiments are provided so that this disclosure will be thorough and complete, and will fully convey the scope of the invention to those skilled in the art. Like numbers refer to like elements throughout. As used herein the term “and/or” includes any and all combinations of one or more of the associated listed items.

The terminology used herein is for the purpose of describing particular embodiments only and is not intended to limit the full scope of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal subparts. As will be understood by one skilled in the art, a range includes each individual member.

### Definitions

### Orthogonal

In the description and claims herein, the term “orthogonal” is used to describe that the valves of the present invention are compressed and delivered at a roughly 90 degree angle compared to traditional transcatheter heart valves. Traditional valves have a central cylinder axis that is parallel to the length-wise axis of the delivery catheter and are deployed from the end of the delivery catheter in a manner akin to pushing a closed umbrella out of a sleeve. The valves of the present invention are compressed and delivered in a sideways manner. Traditional valves can only be expanded as large as what the internal diameter of the delivery catheter will allow. Efforts to increase the expanded diameter of traditional valves have run into the problems of trying to compress too much material and structure into too little space. Mathematically, the term orthogonal refers to an intersecting angle of 90 degrees between two lines or planes. As used, herein the term “substantially orthogonal” refers to an intersecting angle ranging from 75 to 105 degrees. The intersecting angle or orthogonal angle refers to both (i) the relationship between the length-wise cylindrical axis of the delivery catheter and the long-axis of the compressed valve of the invention, where the long-axis is perpendicular to the central cylinder axis of traditional valves, and (ii) the relationship between the long-axis of the compressed or expanded valve of the invention and the axis defined by the blood flow through the prosthetic valve where the blood is flowing, eg. from one part of the body or chamber of the heart to another downstream part of the body or chamber of the heart, such as from an atrium to a ventricle through a native annulus. The intersecting angle may also include 45-135 degrees from normal, i.e. the central axis of the native annulus or the central axis (parallel to blood flow) of the deployed valve.

### Transcatheter

In the description and claims herein, the term “transcatheter” is used to define the process of accessing, controlling, and delivering a medical device or instrument within the lumen of a catheter that is deployed into a heart chamber, as well as an item that has been delivered or controlled by such as process. Transcatheter access is known to include via femoral artery and femoral vein, via brachial artery and vein, via carotid and jugular, via intercostal (rib) space, and via sub-xyphoid. Transcatheter can be synonymous with transluminal and is functionally related to the term “percutaneous” as it relates to delivery of heart valves.

### Tubular Frame

In the description and claims herein, the term “tubular frame”, and also “wire frame” or “flange or “collar” refers to a three-dimensional structural component that is seated within a native valve annulus and is used as a mounting element for a leaflet structure, a flow control component, or a flexible reciprocating sleeve or sleeve-valve, but does not include such a leaflet structure or flow control structure unless further stated.

### Prosthetic Valve Frame

In the description and claims herein, the term “valve frame” or “prosthetic valve frame” or “valve-in-valve” refers to a three-dimensional structural component, usually tubular, cylindrical, or oval or ring-shaped, and that is seated within a native valve annulus and is used as a mounting element for a commercially available valve such as a Sapien®, Sapien 3®, and Sapien XT® from Edwards Lifesciences, the Inspiris Resilia aortic valve from Edwards Lifesciences, the Masters HP 15 mm valve from Abbott, Lotus Edge valve from Boston Scientific, the Crown PRT leaflet structure from Livanova/Sorin, the Carbomedics family of valves from Sorin, or other flow control component, or a flexible reciprocating sleeve or sleeve-valve.

### Valve Frame Structure

The term “frame” as used in the description and claims herein, refers to a flexible structure that is mounted within the lumen of a ring or conduit of native tissue, and that surrounds, encircles, or encloses, in part or wholly, an opening or space. As used herein, a “frame” functions to support a leaflet structure or other flow control structure. The valve frame can be a tube, ring, or cylindrical or conical tube, made from a durable, biocompatible structural material such as Nitinol or similar alloy, wherein the valve frame is formed by manufacturing the structural material as a braided wire frame, a laser-cut wire frame, or a wire loop. The valve frame is about 5-60 mm in height, has an outer diameter dimension, R, of 30-80 mm, and an inner diameter dimension of 31-79 mm, accounting for the thickness of the wire material itself. As stated, the valve frame can have a sideprofile of a tubular shape, a ring shape, cylinder shape, conical tube shape, but may also have a side profile of a flat-cone shape, an inverted flat-cone shape (narrower at top, wider at bottom), a concave cylinder (walls bent in), a convex cylinder (walls bulging out), an angular hourglass, a curved, graduated hourglass, a ring or cylinder having a flared top, flared bottom, or both. In one preferred embodiment, the valve frame used for mounting a prosthetic valve deployed in the tricuspid annulus, and may have a complex shape determined by the anatomical structures where the valve frame is being mounted. For example, in the tricuspid annulus, the circumference of the tricuspid valve may be a rounded ellipse, the septal wall is known to be substantially vertical, and the tricuspid is known to enlarge in disease states along the anterior-posterior line. Accordingly, a prosthetic valve may start in a roughly tubular configuration, and be heat-shaped to provide an upper atrial cuff or flange for atrial sealing and a lower trans-annular tubular section having an hourglass cross-section for about 60-80% of the circumference to conform to the native annulus along the posterior and anterior annular segments while remaining substantially vertically flat along 20-40% of the annular circumference to conform to the septal annular segment.

### Valve Frame Covering

The valve frame is optionally internally or externally covered, partially or completely, with a biocompatible material such as pericardium. The valve frame may also be optionally externally covered, partially or completely, with a second biocompatible material such as polyester or Dacron®.

### Valve Frame Purpose

The valve frame has a central axial lumen where a prosthetic valve or flow-control structure, such as a reciprocating compressible sleeve, is mounted across the diameter of the lumen. The valve frame has an outer circumferential surface for engaging native annular tissue that is also tensioned against the inner aspect of the native annulus and provides structural patency to a weakened native annular ring.

### Valve Frame Optional Collars

The valve frame may optionally have a separate atrial collar attached to the upper (atrial) edge of the frame, for deploying on the atrial floor, that is used to direct blood from the atrium into the sleeve and to seal against blood leakage around the valve frame. The valve frame may also optionally have a separate ventricular collar attached to the lower (ventricular) edge of the frame, for deploying in the ventricle immediately below the native annulus that is used to prevent regurgitant leakage during systole, to prevent dislodging of the device during systole, to sandwich or compress the native annulus or adjacent tissue against the atrial collar, and optionally to attach to and support the sleeve/conduit.

### Valve Frame Delivery

The valve frame may be compressed for transcatheter delivery and may be expandable as a self-expandable shape-memory element or using a transcatheter expansion balloon. Some embodiments may have both an atrial collar and a ventricular collar, whereas other embodiments within the scope of the invention include prosthetic valves having either a single atrial collar, a single ventricular collar, or having no additional collar structure.

### Flow Control Component

In the description and claims herein, the term “flow control component” refers in a non-limiting sense to a leaflet structure having 2-, 3-, 4-leaflets of flexible biocompatible material such a treated or untreated pericardium that is sewn or joined to a tubular frame, to function as a prosthetic valve. Such a valve can be a heart valve, such as a tricuspid, mitral, aortic, or pulmonary, that is open to blood flowing during diastole from atrium to ventricle, and that closes from systolic ventricular pressure applied to the outer surface. Repeated opening and closing in sequence can be described as “reciprocating”.

### Tissue Anchor

In the description and claims herein, the term “tissue anchor” or “plication tissue anchor” or “secondary tissue anchor”, or “dart” or “pin” refers to a fastening device that connects the upper atrial frame to the the native annular tissue, usually at or near the periphery of the collar. The anchor may be positioned to avoid piercing tissue and just rely on the compressive force of the two plate-like collars on the captured tissue, or the anchor, itself or with an integrated securement wire, may pierce through native tissue to provide anchoring, or a combination of both. The anchor may have a specialized securement mechanism, such as a pointed tip with a groove and flanged shoulder that is inserted or popped into a mated aperture or an array of mated apertures that allow the anchor to attach, but prevent detachment when the aperture periphery locks into the groove near the flanged shoulder. The securement wire may be attached or anchored to the collar opposite the pin by any attachment or anchoring mechanisms, including a knot, a suture, a wire crimp, a wire lock having a cam mechanism, or combinations.

### Support Post

The term “support post” refers to a rigid or semi-rigid length of material such as Nitinol or PEEK, that may be mounted on a spoked frame and that runs axially, or down the center of, or within a sewn seam of—, the flexible sleeve. The sleeve may be unattached to the support post, or the sleeve may be directly or indirectly attached to the support post.

In the description that follows, the term “body channel” is used to define a blood conduit or vessel within the body. Of course, the particular application of the prosthetic heart valve determines the body channel at issue. An aortic valve replacement, for example, would be implanted in, or adjacent to, the aortic annulus. Likewise, a tricuspid or mitral valve replacement will be implanted at the tricuspid or mitral annulus. Certain features of the present invention are particularly advantageous for one implantation site or the other. However, unless the combination is structurally impossible, or excluded by claim language, any of the heart valve embodiments described herein could be implanted in any body channel.

The term “lumen” refers to the inside of the cylinder tube. The term “bore” refers to the inner diameter.

Displacement—the volume of fluid displaced by one complete stroke or revolution

Ejection fraction is a measurement of the percentage of blood leaving your heart each time it contracts. During each heartbeat pumping cycle, the heart contracts and relaxes. When your heart contracts, it ejects blood from the two pumping chambers (ventricles)

As a point of further definition, the term “expandable” is used herein to refer to a component of the heart valve capable of expanding from a first, delivery diameter to a second, implantation diameter. An expandable structure, therefore, does not mean one that might undergo slight expansion from a rise in temperature, or other such incidental cause. Conversely, “non-expandable” should not be interpreted to mean completely rigid or a dimensionally stable, as some slight expansion of conventional “non-expandable” heart valves, for example, may be observed.

Force—A push or pull acting upon a body. In a hydraulic cylinder, it is the product of the pressure on the fluid, multiplied by the effective area of the cylinder piston.

### Prosthetic Valve

The term “valve prosthesis” or “prosthetic valve” refers to a combination of a frame and a leaflet or flow control structure, and encompasses both complete replacement of an anatomical part, e.g. a new mechanical valve replaces a native valve, as well as medical devices that take the place of and/or assist, repair, or improve existing anatomical parts, e.g. native valve is left in place. For mounting within a passive assist cage, the invention contemplates a wide variety of (bio)prosthetic artificial heart valves. Contemplated as within the scope of the invention are ball valves (e.g. Starr-Edwards), bileaflet valves (St. Jude), tilting disc valves (e.g. Bjork-Shiley), stented pericardium heart-valve prosthesis' (bovine, porcine, ovine) (Edwards line of bioprostheses, St. Jude prosthetic valves), as well as homograft and autograft valves. For bioprosthetic pericardial valves, it is contemplated to use bioprosthetic aortic valves, bioprosthetic mitral valves, bioprosthetic tricuspid valves, and bioprosthetic pulmonary valves.

### Frame Material

Preferably, the frame is made from superelastic metal wire, such as Nitinol™ wire or other similarly functioning material. The material may be used for the frame/stent, for the collar, and/or for anchors. It is contemplated as within the scope of the invention to use other shape memory alloys such as Cu—Zn—Al—Ni alloys, Cu—Al—Ni alloys, as well as polymer composites including composites containing carbon nanotubes, carbon fibers, metal fibers, glass fibers, and polymer fibers. It is contemplated that the frame may be constructed as a braid, wire, or laser cut wire frame. Such materials are available from any number of commercial manufacturers, such as Pulse Systems. Laser cut wire frames are preferably made from Nickel-Titanium (Nitinol™), but also without limitation made from stainless steel, cobalt chromium, titanium, and other functionally equivalent metals and alloys, or Pulse Systems braided frame that is shape-set by heat treating on a fixture or mandrel.

One key aspect of the frame design is that it be compressible and when released have the stated property that it return to its original (uncompressed) shape. This requirement limits the potential material selections to metals and plastics that have shape memory properties. With regards to metals, Nitinol has been found to be especially useful since it can be processed to be austenitic, martensitic or super elastic. Martensitic and super elastic alloys can be processed to demonstrate the required compression features.

### Laser Cut

One possible construction of the wire frame envisions the laser cutting of a thin, isodiametric Nitinol tube. The laser cuts form regular cutouts in the thin Nitinol tube. In one preferred embodiment, the Nitinol tube expands to form a three-dimensional structure formed from diamond-shaped cells. The structure may also have additional functional elements, e.g. loops, anchors, etc. for attaching accessory components such as biocompatible covers, tissue anchors, releasable deployment and retrieval control guides, knobs, attachments, rigging, and so forth.

Secondarily the tube is placed on a mold of the desired shape, heated to the Martensitic temperature and quenched. The treatment of the wire frame in this manner will form a device that has shape memory properties and will readily revert to the memory shape at the calibrated temperature.

### Braided Wire

A frame can be constructed utilizing simple braiding techniques. Using a Nitinol wire—for example a 0.012″ wire—and a simple braiding fixture, the wire is wound on the braiding fixture in a simple over/under braiding pattern until an isodiametric tube is formed from a single wire. The two loose ends of the wire are coupled using a stainless steel or Nitinol coupling tube into which the loose ends are placed and crimped. Angular braids of approximately 60 degrees have been found to be particularly useful. Secondarily, the braided wire frame is placed on a shaping fixture and placed in a muffle furnace at a specified temperature to set the wire frame to the desired shape and to develop the martensitic or super elastic properties desired.

Tethers—The tethers are made from surgical-grade materials such as biocompatible polymer suture material. Non-limiting examples of such material include ultra high-molecular weight polyethylene (UHMWPE), 2-0 exPFTE(polytetrafluoroethylene) or 2-0 polypropylene. In one embodiment the tethers are inelastic. It is also contemplated that one or more of the tethers may optionally be elastic to provide an even further degree of compliance of the valve during the cardiac cycle.

### Tines-Anchors-Tines/Barbs

The device can be seated within the valvular annulus through the use of tines or barbs. These may be used in conjunction with, or in place of one or more tethers. The tines or barbs are located to provide attachment to adjacent tissue. Tines are forced into the annular tissue by mechanical means such as using a balloon catheter. In one non-limiting embodiment, the tines may optionally be semi-circular hooks that upon expansion of the wire frame body, pierce, rotate into, and hold annular tissue securely. Anchors are deployed by over-wire delivery of an anchor or anchors through a delivery catheter. The catheter may have multiple axial lumens for delivery of a variety of anchoring tools, including anchor setting tools, force application tools, hooks, snaring tools, cutting tools, radio-frequency and radiological visualization tools and markers, and suture/thread manipulation tools. Once the anchor(s) are attached to the moderator band, tensioning tools may be used to adjust the length of tethers that connect to an implanted valve to adjust and secure the implant as necessary for proper functioning. It is also contemplated that anchors may be spring-loaded and may have tether-attachment or tether-capture mechanisms built into the tethering face of the anchor(s). Anchors may also have in-growth material, such as polyester fibers, to promote in-growth of the anchors into the myocardium.

In one embodiment, a prosthetic valve frame may include an atrial collar, a ventricular collar, or both.

### Cover Material-Biological Tissue-

The tissue used herein is a biological tissue that is a chemically stabilized pericardial tissue of an animal, such as a cow (bovine pericardium) or sheep (ovine pericardium) or pig (porcine pericardium) or horse (equine pericardium). Preferably, the tissue is bovine pericardial tissue. Examples of suitable tissue include that used in the products Duraguard®, Peri-Guard®, and Vascu-Guard®, all products currently used in surgical procedures, and which are marketed as being harvested generally from cattle less than 30 months old. Other patents and publications disclose the surgical use of harvested, biocompatible animal thin tissues suitable herein as biocompatible “jackets” or sleeves for implantable stents, including for example, U.S. Pat. No. 5,554,185 to Block, U.S. Pat. No. 7,108,717 to Design & Performance-Cyprus Limited disclosing a covered stent assembly, U.S. Pat. No. 6,440,164 to Scimed Life Systems, Inc. disclosing a bioprosthetic valve for implantation, and U.S. Pat. No. 5,336,616 to LifeCell Corporation discloses acellular collagen-based tissue matrix for transplantation.

### Polymers

In one preferred embodiment, the conduit may optionally be made from a synthetic material such a polyurethane or polytetrafluoroethylene.

Where a thin, durable synthetic material is contemplated, e.g. for a covering, synthetic polymer materials such expanded polytetrafluoroethylene or polyester may optionally be used. Other suitable materials may optionally include thermoplastic polycarbonate urethane, polyether urethane, segmented polyether urethane, silicone polyether urethane, silicone-polycarbonate urethane, and ultra-high molecular weight polyethylene. Additional biocompatible polymers may optionally include polyolefins, elastomers, polyethylene-glycols, polyethersulphones, polysulphones, polyvinylpyrrolidones, polyvinylchlorides, other fluoropolymers, silicone polyesters, siloxane polymers and/or oligomers, and/or polylactones, and block co-polymers using the same.

### Polyamides (PA)

PA is an early engineering thermoplastic invented that consists of a “super polyester” fiber with molecular weight greater than 10,000. It is commonly called Nylon. Application of polyamides includes transparent tubing's for cardiovascular applications, hemodialysis membranes, and also production of percutaneous transluminal coronary angioplasty (PTCA) catheters.

### Polyolefin

Polyolefins include polyethylene and polypropylene are the two important polymers of polyolefins and have better biocompatibility and chemical resistance. In cardiovascular uses, both low-density polyethylene and high-density polyethylene are utilized in making tubing and housings. Polypropylene is used for making heart valve structures.

### Polyesters

Polyesters includes polyethylene-terephthalate (PET), using the name Dacron. It is typically used as knitted or woven fabric for vascular grafts. Woven PET has smaller pores which reduces blood leakage and better efficiency as vascular grafts compared with the knitted one. PET grafts are also available with a protein coating (collagen or albumin) for reducing blood loss and better biocompatibility [39]. PET vascular grafts with endothelial cells have been searched as a means for improving patency rates. Moreover, polyesters are widely preferred material for the manufacturing of bioabsorbable stents. Poly-L-lactic acids (PLLA), polyglycolic acid (PGA), and poly(D, L-lactide/glycolide) copolymer (PDLA) are some of the commonly used bioabsorbable polymers.

### Polytetrafluoroethylene

Polytetrafluoroethylene (PTFE) is synthetic fluorocarbon polymer with the common commercial name of Teflon by Dupont Co. Common applications of PTFE in cardiovascular engineering include vascular grafts and heart valves. PTFE sutures are used in the repair of mitral valve for myxomatous disease and also in surgery for prolapse of the anterior or posterior leaflets of mitral valves. PTFE is particularly used in implantable prosthetic heart valve rings. It has been successfully used as vascular grafts when the devices are implanted in high-flow, largediameter arteries such as the aorta. Problem occurs when it is implanted below aortic bifurcations and another form of PTFE called elongated-PTFE (e-PTFE) was explored. Expanded PTFE is formed by compression of PTFE in the presence of career medium and finally extruding the mixture. Extrudate formed by this process is then heated to near its glass transition temperature and stretched to obtain microscopically porous PTFE known as e-PTFE. This form of PTFE was indicated for use in smaller arteries with lower flow rates promoting low thrombogenicity, lower rates of restenosis and hemostasis, less calcification, and biochemically inert properties.

### Polyurethanes

Polyurethane has good physiochemical and mechanical properties and is highly biocompatible which allows unrestricted usage in blood contacting devices. It has high shear strength, elasticity, and transparency. Moreover, the surface of polyurethane has good resistance for microbes and the thrombosis formation by PU is almost similar to the versatile cardiovascular biomaterial like PTFE. Conventionally, segmented polyurethanes (SPUs) have been used for various cardiovascular applications such as valve structures, pacemaker leads and ventricular assisting device.

### Covered Wire Frame Materials

Drug-eluting wire frames are contemplated for use herein. DES basically consist of three parts: wire frame platform, coating, and drug. Some of the examples for polymer free DES are Amazon Pax (MINVASYS) using Amazonia CroCo (L605) cobalt chromium (Co—Cr) wire frame with Paclitaxel as an antiproliferative agent and abluminal coating have been utilized as the carrier of the drug. BioFreedom (Biosensors Inc.) using stainless steel as base with modified abluminal coating as carrier surface for the antiproliferative drug Biolimus A9. Optima (CID S.r.I.) using 316 L stainless steel wire frame as base for the drug Tacrolimus and utilizing integrated turbostratic carbofilm as the drug carrier. VESTA sync (MIV Therapeutics) using GenX stainless steel (316 L) as base utilizing microporous hydroxyapatite coating as carrier for the drug Sirolimus. YUKON choice (Translumina) used 316 L stainless steel as base for the drugs Sirolimus in combination with Probucol.

Biosorbable polymers may also be used herein as a carrier matrix for drugs. Cypher, Taxus, and Endeavour are the three basic type of bioabsorbable DES. Cypher (J&J, Cordis) uses a 316 L stainless steel coated with polyethylene vinyl acetate (PEVA) and poly-butyl methacrylate (PBMA) for carrying the drug Sirolimus. Taxus (Boston Scientific) utilizes 316 L stainless steel wire frames coated with translute Styrene Isoprene Butadiene (SIBS) copolymer for carrying Paclitaxel which elutes over a period of about 90 days. Endeavour (Medtronic) uses a cobalt chrome driver wire frame for carrying zotarolimus with phosphorylcholine as drug carrier. BioMatrix employing S-Wire frame (316 L) stainless steel as base with polylactic acid surface for carrying the antiproliferative drug Biolimus. ELIXIR-DES program (Elixir Medical Corp) consisting both polyester and polylactide coated wire frames for carrying the drug novolimus with cobalt-chromium (Co—Cr) as base. JACTAX (Boston Scientific Corp.) utilized D-lactic polylactic acid (DLPLA) coated (316 L) stainless steel wire frames for carrying Paclitaxel. NEVO (Cordis Corporation, Johnson & Johnson) used cobalt chromium (Co—Cr) wire frame coated with polylactic-co-glycolic acid (PLGA) for carrying the drug Sirolimus.

Examples of preferred embodiments of the reciprocating pressure conduit valve include the following details and features.

### EXAMPLE

One preferred embodiment of the present invention is exemplified by a compressible bileaflet frame as a flow control component for a side delivered transcatheter prosthetic valve, comprising: (i) an annular ring having a distal strut and a proximal strut opposite the distal strut, and (ii) a first leaflet and a second leaflet disposed within the annular ring forming a reciprocating flow control component, each leaflet comprised of pericardium or a biocompatible material. The annular ring comprises a first semi-ellipsoidal frame member and a second semi-ellipsoidal frame member or may be unitary in construction (a single 3D manufacture), each semi-ellipsoidal frame having a distal end and a proximal end, wherein the distal end of the first semi-ellipsoidal frame member and the second semi-ellipsoidal are adjacent, and wherein the proximal end of the first semi-ellipsoidal frame member and the second semi-ellipsoidal are adjacent. The distal strut and proximal strut are each comprised of a first distal band/paddle/post attached length-wise to a second distal band/paddle/post. The distal strut and the proximal strut are oriented in an uncompressed configuration at an angle of between 45-135 degrees to a plane of the annular ring. The leaflets are attached along a upper portion to the semi-ellipsoidal frame members, and attached at their distal portions to the distal band/paddle/post and at their proximal portions to the proximal band/paddle/post. The annular ring has a diameter of about 25-80 mm and the distal and proximal struts have a length of about 5-60 mm.

### Example

One preferred embodiment of a side delivered transcatheter prosthetic valve frame has a tubular frame, wherein the valve frame is compressible to a compressed configuration for introduction into the body using a delivery catheter for implanting at a desired location in the body, said compressed configuration having a long-axis oriented at an intersecting angle of between 45-135 degrees to the central, cylindrical axis of the native annulus, and expandable to an expanded configuration having a long-axis oriented at an intersecting angle of between 45-135 degrees to the central, cylindrical axis of the native annulus, wherein the long-axis of the compressed configuration of the valve is substantially parallel to a length-wise cylindrical axis of the delivery catheter, wherein the valve has a height of about 5-60 mm and a diameter of about 25-80 mm.

This heart valve frame can be delivered to the heart using the inferior vena cava (IVC/femoral transcatheter delivery pathway compressed within a catheter, and expelled from the catheter to be deployed without open heart surgery.

### Example

In another preferred embodiment of a transcatheter valve frame, comprises: a cylindrical tubular frame having a height of about 5-60 mm and an outer diameter of about 25-80 mm, said tubular frame comprised of a braid, wire, or laser-cut wire frame having a substantially circular central aperture, said tubular frame partially covered with a biocompatible material; an upper tension arm attached to a distal upper edge of the tubular frame, the upper tension arm comprised of stent, segment of tubular frame, wire loop or wire frame extending from about 10-30 mm away from the tubular frame; a lower tension arm extending from a distal side of the tubular frame, the lower tension arm comprised of stent, segment of tubular frame, wire loop or wire frame extending from about 10-40 mm away from the tubular frame; and at least one tissue anchor to connect the tubular frame to native tissue.

### Example

In a preferred embodiment of the invention, there is also provided a method for orthogonal delivery of implantable prosthetic valve frame to a desired location in the body, the method comprising the steps: (i) advancing a delivery catheter to the desired location in the body and delivering an expandable prosthetic valve frame to the desired location in the body by releasing the valve frame from the delivery catheter, wherein the valve frame is compressible to a compressed configuration for introduction into the body using a delivery catheter for implanting at a desired location in the body, said compressed configuration having a long-axis oriented at an intersecting angle of between 45-135 degrees to the central, cylindrical axis of the native annulus, and expandable to an expanded configuration having a long-axis oriented at an intersecting angle of between 45-135 degrees to the central, cylindrical axis of the native annulus, wherein the long-axis of the compressed configuration of the valve frame is substantially parallel to a length-wise cylindrical axis of the delivery catheter, wherein the valve frame has a height of about 5-60 mm and a diameter of about 25-80 mm.

### Example

In a preferred embodiment of the invention, there is also provided a method for orthogonally (side) loading an implantable prosthetic valve frame into a delivery catheter, the method comprising the steps: loading an implantable prosthetic valve frame sideways into a tapering fixture or funnel attached to a delivery catheter, wherein the valve frame is compressible to a compressed configuration for introduction into the body using a delivery catheter for implanting at a desired location in the body, said compressed configuration having a long-axis oriented at an intersecting angle of between 45-135 degrees to the central, cylindrical axis of the native annulus, and expandable to an expanded configuration having a long-axis oriented at an intersecting angle of between 45-135 degrees to the central, cylindrical axis of the native annulus, wherein the long-axis of the compressed configuration of the valve frame is substantially parallel to a length-wise cylindrical axis of the delivery catheter, wherein the valve frame has a height of about 5-60 mm and a diameter of about 25-80 mm.

### Example

The transcatheter prosthetic heart valve may be percutaneously delivered using a transcatheter process via the femoral through the inferior vena cava (IVC), superior vena cava (SVC), jugular vein, brachial vein, sub-xyphoid, intercostal access across the chest wall, and trans-septal through the fossa ovalis.

The device is delivered via catheter to the right or left atrium and is expanded from a compressed shape that fits with the internal diameter of the catheter lumen. The compressed valve is loaded external to the patient into the delivery catheter, and is then pushed out of the catheter when the capsule arrives to the atrium. The cardiac treatment technician visualizes this delivery using available imaging techniques such as fluoroscopy or ultrasound, and in a preferred embodiment the valve frame self-expands upon release from the catheter since it is constructed in part from shape-memory material, such as Nitinol®, a nickel-titanium alloy used in biomedical implants.

In another embodiment, the valve frame may be constructed of materials that requires balloon-expansion after the capsule has been ejected from the catheter into the atrium.

The atrial collar/frame is expanded to their functional diameter, and deployed into the native annulus, providing a radial tensioning force to secure the valve frame. Once the frame is deployed about the tricuspid annulus, fasteners secure the device about the native annulus. Additional fastening of the device to native structures may be performed, and the deployment is complete. Further adjustments using hemodynamic imaging techniques are contemplated as within the scope of the invention in order to ensure the device is secure, is located and oriented as planned, and is functioning.

### VVDYNE-13 PARTS LIST

102 bileaflet frame
104 two-piece annular ring
106 distal strut
108 proximal strut
110 first leaflet
112 second leaflet
114 first semi-ellisoidal frame member of annular ring
116 second semi-ellisoidal frame member of annular ring
118 distal end of first semi-ellisoidal frame member of annular ring
120 proximal end of first semi-ellisoidal frame member of annular ring
122 distal end of second semi-ellisoidal frame member of annular ring
124 proximal end of second semi-ellisoidal frame member of annular ring
126 first distal post of distal strut
128 second distal post of distal strut
130 first proximal post of proximal strut
132 second proximal post of proximal strut
134 upper leaflet attachment portion of first semi-ellisoidal frame member
136 upper leaflet attachment portion of second semi-ellisoidal frame member
138 square annular frame
140 first square member
142 second square member
150 coaptation spacer
152 coaptation flare
154 leaflet free edge
202 side delivered heart valve
204 a self-expanding annular support frame,
206 a central channel
208 an outer perimeter wall
210 a central vertical axis
212 atrial collar
214 a front wall portion of perimeter wall
216 a back wall portion
218 a proximal side
220 a proximal fold area
222 a distal side
224 a distal fold area
226 a front upper collar portion of front wall
228 a front lower body portion of front wall
230 a back upper collar portion of back wall
232 a back lower body portion
234 a flow control component mounted within the annular support frame
236 horizontal axis
238 compressible wire cells
240 frame inner surface
242 frame outer surface
244 biocompatible cover
246 rigid batons or panels
248 RVOT tab/distal sub annular tab
250 upper tension arm
252 proximal sub annular tab
254 tissue anchor
256 compressed configuration
258 partial expanded configuration
260 expanded configuration
270 delivery catheter
272 deployment tool

### DRAWINGS

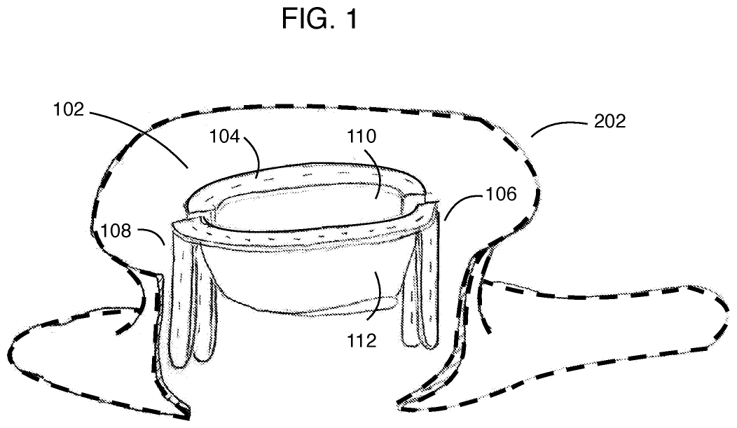

Referring now to the drawings, FIG. 1 is an illustration of a front side (plan) view, with a cut-away view of a leaflet frame 102 as a flow control component according to the present invention shown mounted within a side delivered transcatheter prosthetic heart valve 202. Leaflet frame 102 is comprised of an annular ring 104 with a vertical distal post 106 and a vertical proximal post 108. Leaflet frame 102 has a first leaflet 110 and a second leaflet 112 that are attached to the frame leaving a lower free edge of the leaflet(s) free to coapt and open and close in a reciprocal manner to provide a flow control component to allow blood to flow thru (top to bottom) and to block blood flow in the opposite direction.

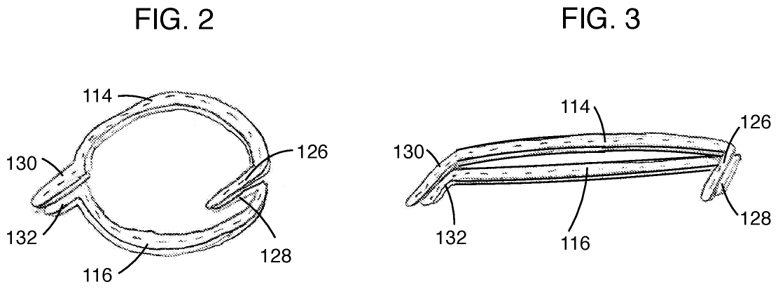

FIG. 2 is an illustration of a perspective detail view from below, of an annular ring 104 and struts 106, 108 of a leaflet frame 102 in an expanded configuration of a flow control component. FIG. 2 shows that annular ring 104 may be constructed in two pieces. A first rounded or semi-ellipsoidal frame member 114 and a second rounded or semi-ellipsoidal frame member 116 form the annular ring 104. Similarly, distal strut 106 is formed from a pairing of the a first distal post 126 and a second distal post 128, and proximal strut 108 is formed from mating or pairing a first proximal post 130 and second proximal post 132.

In some embodiments, the annular ring and struts may have a unitary construction. In other embodiments, the annular ring and struts may be constructed in two or more components for ease of attachment of the leaflets.

FIG. 3 is an illustration of a perspective view an annular ring and struts of a leaflet frame in a flattened compressed (rollable) configuration of a flow control component according to the present invention. FIG. 3 shows annular ring 104 and struts 106, 108 of a leaflet frame 102 in a compressed or flattened configuration of a flow control component. FIG. 3 shows that annular ring 104 may be constructed in two pieces, and in a compressed configuration, the first rounded or semi-ellipsoidal frame member 114 and the second rounded or semi-ellipsoidal frame member 116 may lay flat and be adjacent to one another.

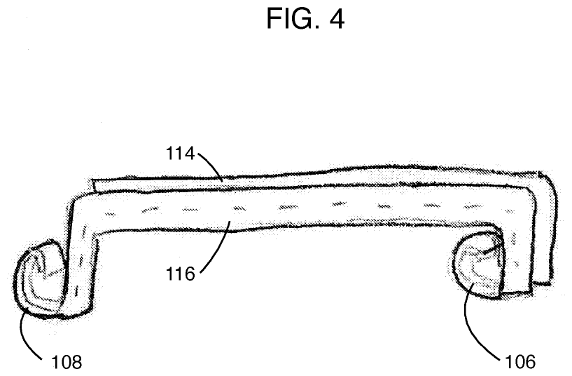

FIG. 4 is an illustration of a front plan view, of an annular ring 104 and struts 106, 108 of a leaflet frame 102 in a flattened compressed (rollable) configuration of a flow control component showing the annular ring 114, 116 flattened and the struts 106, 108 in partial roll-up compression.

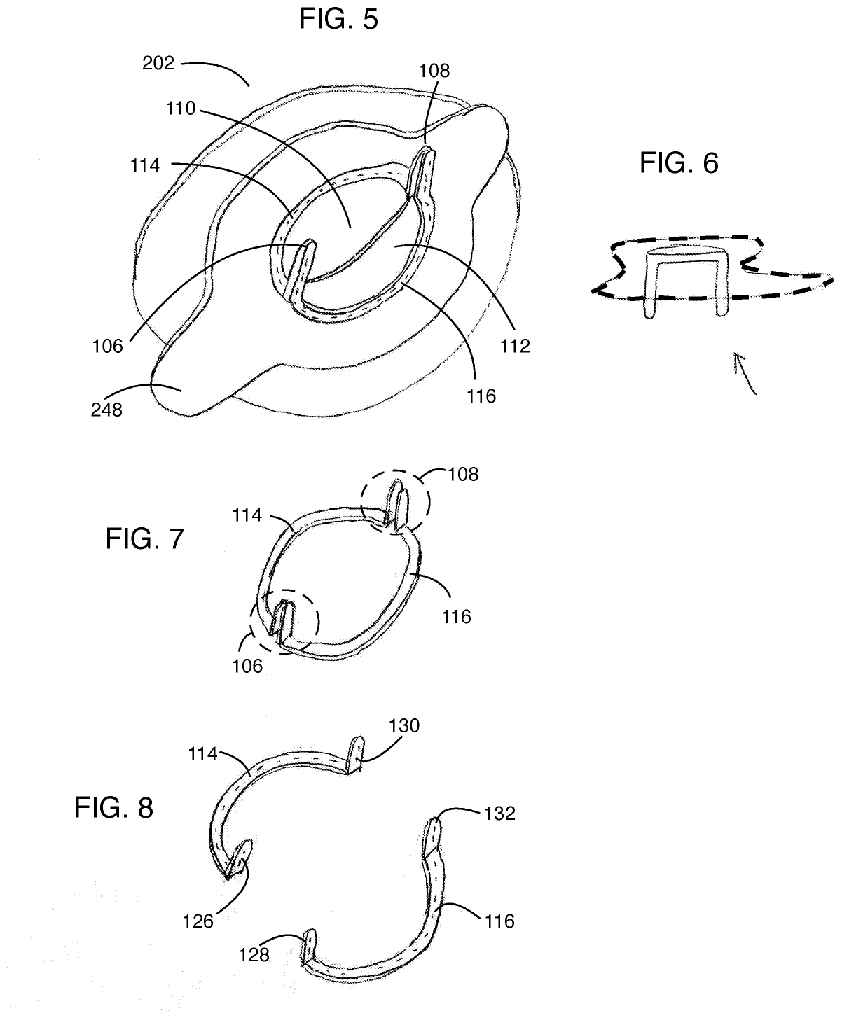

FIG. 5 is an illustration of a perspective view from below, of a leaflet frame 102 with leaflets 110, 112 as a a flow control component in a side delivered transcatheter prosthetic heart valve 202 according to the present invention having a braid or laser-cut construction for the tubular frame, with an extended tab or tension arm 248 that extends away from the tubular frame.

FIG. 6 is an illustration of a side view of a heart valve outline with a position of the leaflet frame 102 according to the present invention.

FIG. 7 is an illustration of an inverted perspective view, of an annular ring 104 and struts 106, 108 of a leaflet frame 102 as a flow control component in an expanded configuration. First frame member 114 and second frame member 116 are show forming the annular ring 104.

FIG. 8 is an illustration of an inverted perspective view, of an annular ring 104 and struts 106, 108 of a leaflet frame 102 as a flow control component in an expanded configuration. First frame member 114 and second frame member 116 are show forming the annular ring 104, and distal posts 126, 128 are shown forming distal strut 106, with proximal posts 103, 132 shown forming proximal strut 108.

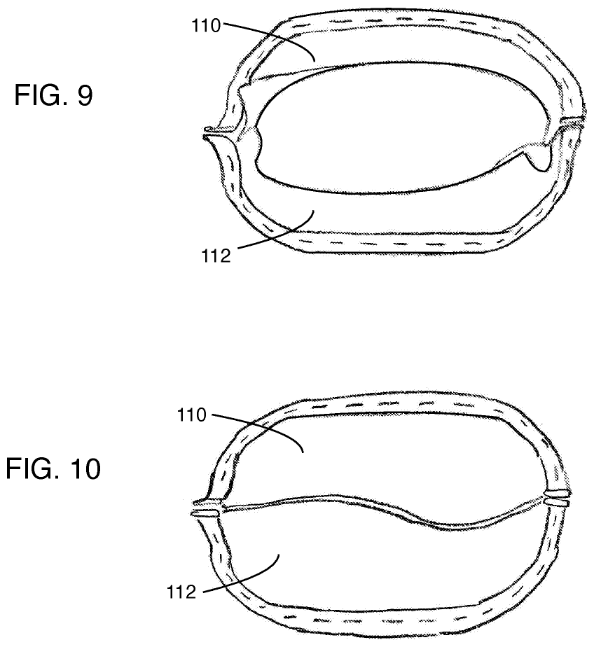

FIG. 9 is an illustration of a bottom view of a leaflet frame according to the present invention with annular ring, struts, and leaflets 110, 112 as a flow control component with leaflets 110, 112 in an open leaflet configuration.

FIG. 10 is an illustration of a bottom view of a leaflet frame according to the present invention with annular ring, struts, and leaflets 110, 112 as a flow control component with leaflets 110, 112 in a closed leaflet configuration.

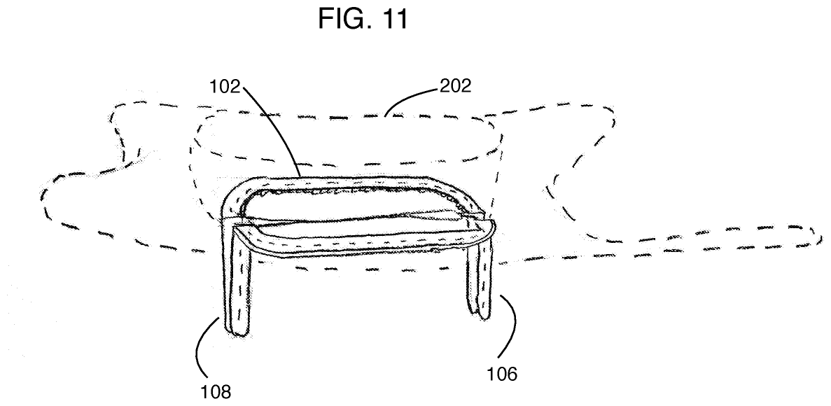

FIG. 11 is an illustration of a front view of a leaflet frame 102 with annular ring, struts, and leaflets as a flow control component in a closed leaflet configuration with an outline of a side-delivered valve 202, and showing the leaflet frame positioned in a lower aspect of the valve body with struts 106, 108 extending ventricularly.

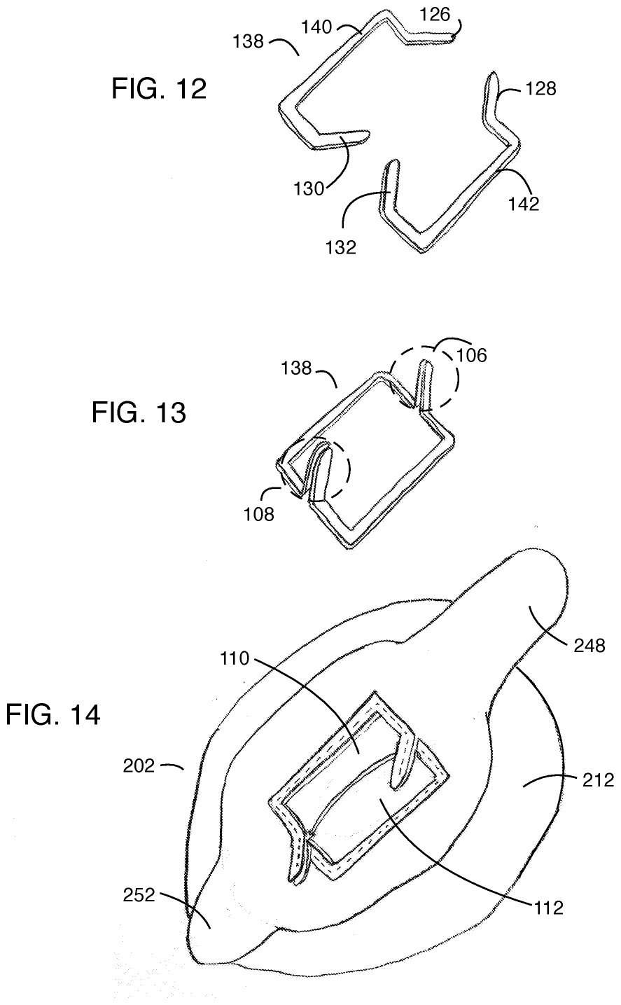

FIG. 12 is an illustration of an inverted exploded view of a square annular frame 138 with first and second square members 140, 142 and struts 106, 108 comprised of distal posts 126, 128 and proximal posts 130, 132, according to the present invention of a rectilinear leaflet frame as a flow control component in an expanded configuration.

FIG. 13 is an illustration of an inverted view of a bileaflet frame 102 having a square annular frame 104 and struts 106, 108 of a rectilinear leaflet frame as a flow control component in an expanded configuration showing struts aligned and connected length-wise.

FIG. 14 is an illustration of a perspective view from below of a leaflet frame 102 with leaflets 110, 112 as a flow control component in a side delivered transcatheter prosthetic heart valve 202 with collar 212 according to the present invention. In some embodiments, valve 202 and/or leaflet frame 102 may have a braid or laser-cut construction. The valve 202 is shown having a distal sub-annular anchor tab or tension arm 248 and a proximal subannular anchor tab 252, that each extends away from the tubular frame distally and proximally, respectively.

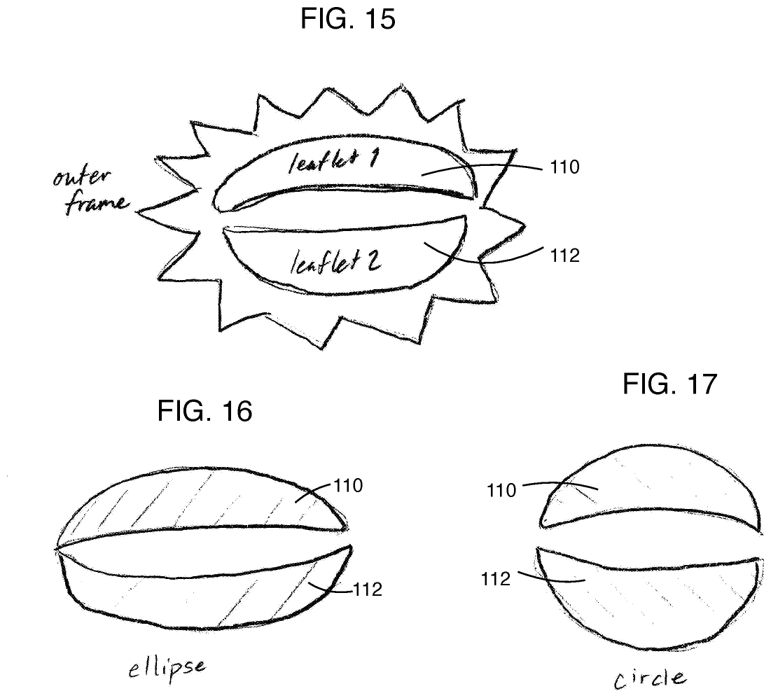

FIG. 15 is an illustration of a top view of a valve according to the present invention showing position of the leaflets 110, 112 in relation to the outer frame.

FIG. 16 is an illustration of a top view of an ellipse configuration of bileaflets 110, 112 according to the present invention.

FIG. 17 is an illustration of a top view of a circular ellipse configuration of bileaflets 110, 112.

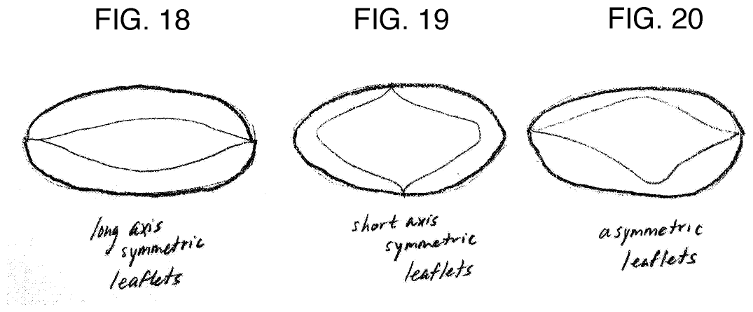

FIGS. 18-20 show various embodiments of configurations of the leaflets. FIG. 18 is an illustration of a top view of a bi-leaflet pair in a long axis symmetric configuration. FIG. 19 is an illustration of a top view of a bi-leaflet pair in a short axis symmetric configuration. FIG. 20 is an illustration of a top view of a bi-leaflet pair in an asymmetric configuration.

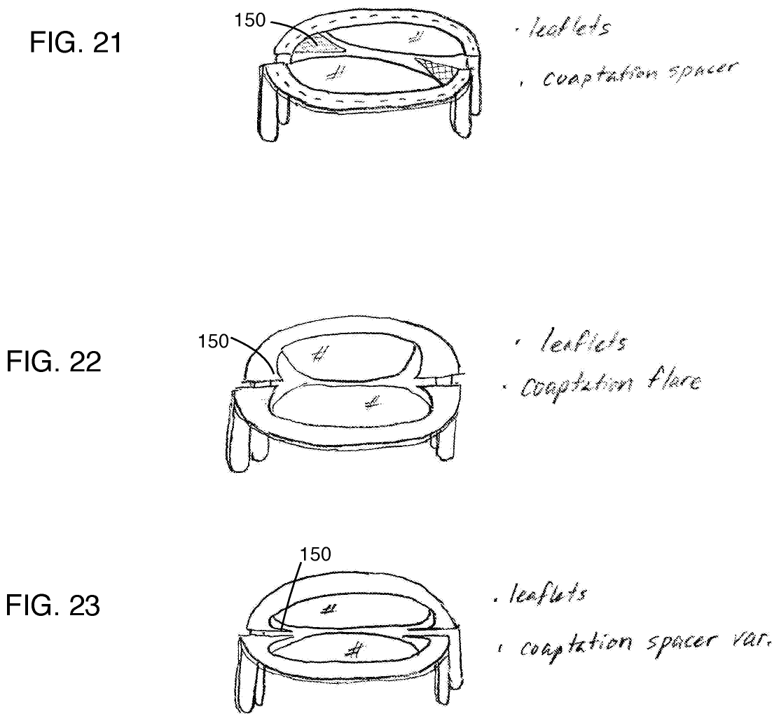

FIGS. 21-23 show various embodiments of coaptation devices, such as spacers or flared portions of the leaflet frame. FIG. 21 is an illustration of a front perspective view of an annular ring with coaptation spacers 150 providing improved leaflet sealing of a leaflet frame as a flow control component in an expanded uncompressed (rollable) configuration showing struts aligned length-wise. FIG. 22 is an illustration of a front perspective view of an annular ring with flared coaptation spacers 150 providing improved leaflet sealing of a leaflet frame as a flow control component in an expanded uncompressed (rollable) configuration showing struts aligned length-wise. FIG. 23 is an illustration of a front perspective view of an annular ring with split-flared coaptation spacers 150 providing improved leaflet sealing of a leaflet frame as a flow control component in an expanded uncompressed (rollable) configuration showing struts aligned length-wise.

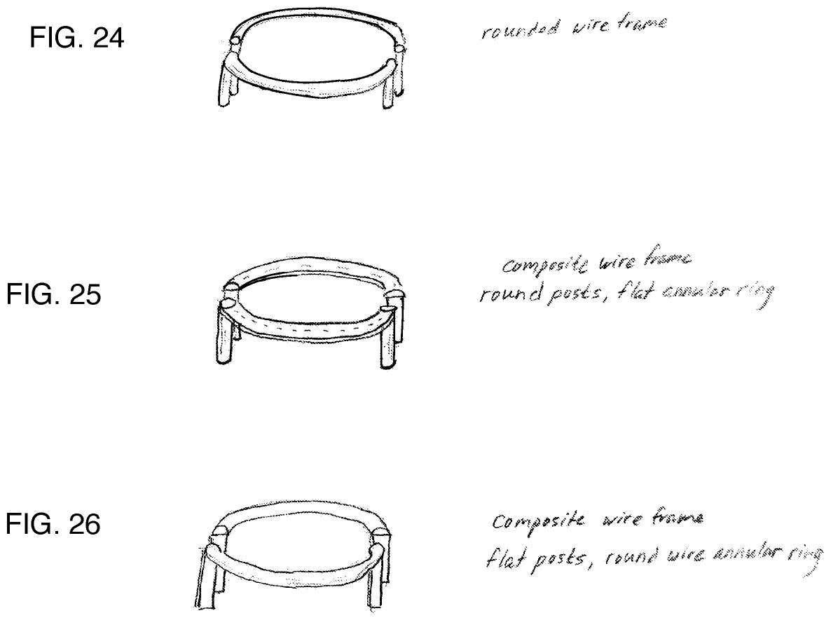

FIG. 24 is an illustration of a front perspective view of a variation of an annular ring made from a non-planar structure, e.g. rounded structure or wire, for a leaflet frame as a flow control component in an expanded uncompressed (rollable) configuration.

FIG. 25 is an illustration of a front perspective view of a composite variation of a leaflet frame having a flat band annular ring and round wire struts as a flow control component in an expanded uncompressed (rollable) configuration.

FIG. 26 is an illustration of a front perspective view of a composite variation of a leaflet frame having a non-planar annular ring e.g. rounded, semi-round, or wire, and flat band struts as a flow control component in an expanded uncompressed (rollable) configuration.

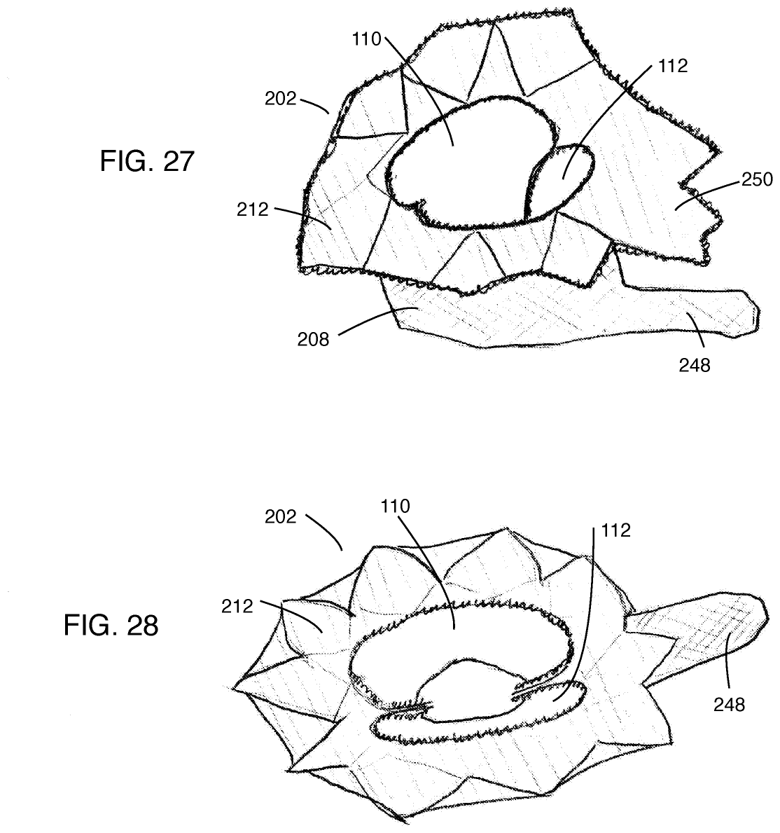

FIG. 27 is an illustration of a front perspective view of a valve prosthesis 202 having a bi-leaflet frame 102 having leaflets 110, 112 mounted within a central aperture 206 of an atrial collar 212 attached to a valve body portion 208 and having an RVOT tab 248 extending from the lower part of the valve body portion, and an upper tension member 250 extending from a distal edge of the collar 212.

FIG. 28 is an illustration of a top view of a valve prosthesis 202 having a bileaflet frame 102 having leaflets 110, 112 as a flow control component mounted within a central aperture of an atrial collar 212 attached to a valve body portion 208 and having an RVOT tab 248 extending from the lower part of the valve body portion.

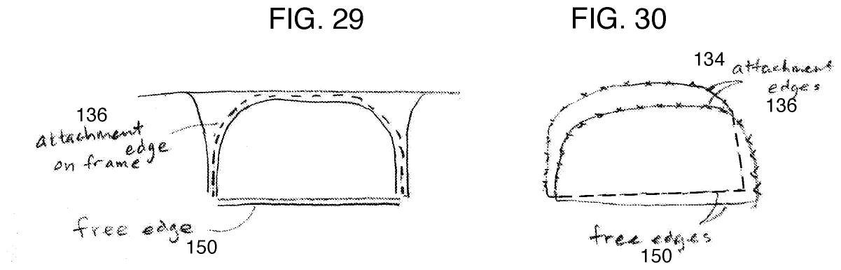

FIG. 29 is an illustration of a side view of a valve prosthesis having a leaflet frame with atrial collar attached to a valve body portion and showing leaflet attachment on a frame edge 136 and leaflet free edge 150.

FIG. 30 is an illustration of a side view of a pair of leaflets and shows a parabolic shape with attachment edges 134, 136 along the top and side edges, and a free (coaptation) edge 150 along the bottom edge.

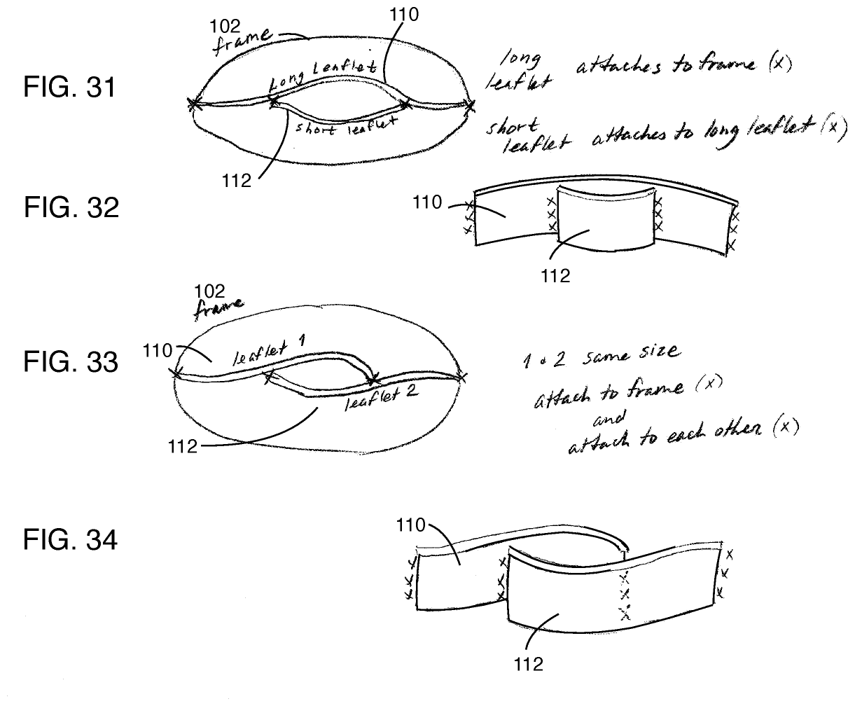

FIG. 31 is an illustration of a top view of a frame 102 having a pair of leaflets 110, 112, one long 110, one short 112, in one embodiment according to the present invention.

FIG. 32 is an illustration of a perspective view of a pair of leaflets sans frame, one long 110, one short 112, in one embodiment according to the present invention.

FIG. 33 is an illustration of top view of a frame 102 having a pair of leaflets 110, 112, equal in lateral length and arranged as interconnected S shapes, in one embodiment according to the present invention.

FIG. 34 is an illustration of a perspective view of a pair of leaflets 110, 112, san frame, equal in lateral length and arranged as interconnected S shapes, in one embodiment according to the present invention.

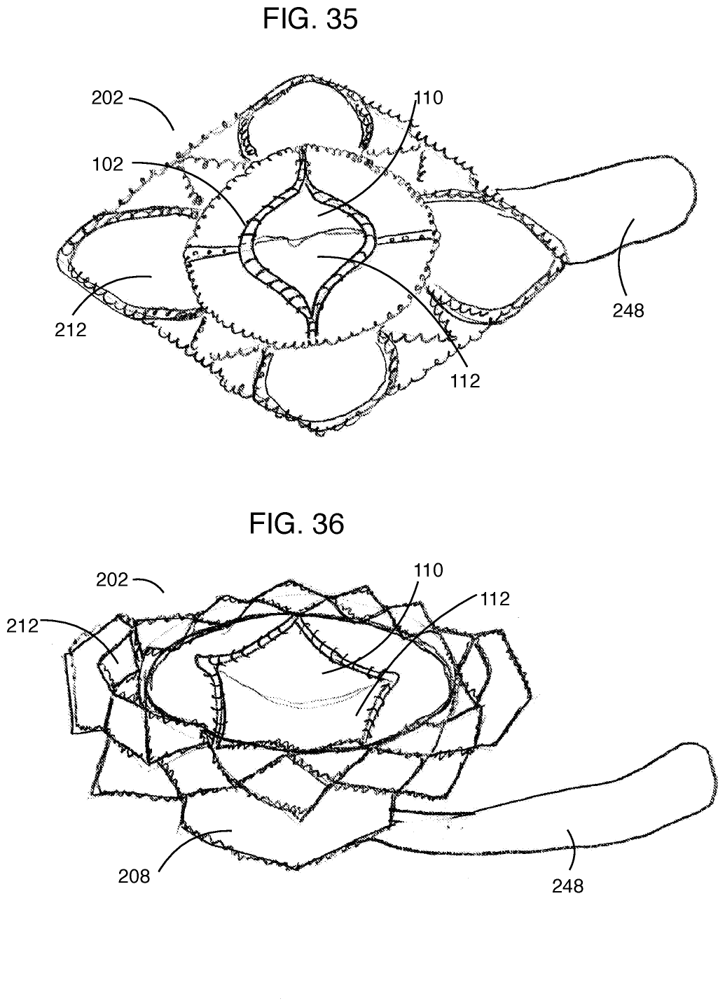

FIG. 35 is an illustration of a top view of a valve prosthesis 202 having a bileaflet frame 102 as a flow control component mounted along a short axis—septal to anterior—within a central aperture of an atrial collar 212 attached to a valve body portion and having an RVOT tab 248 extending from the lower part of the valve body portion.

FIG. 36 is an illustration of a top perspective view of a valve prosthesis 202 having a bi-leaflet frame 102 as a flow control component mounted along a long axis—distal (pulmonary artery) to proximal (inferior vena cava)—within a central aperture of an atrial collar 212 attached to a valve body portion 208 and having an RVOT tab 248 extending from the lower part of the valve body portion 208.

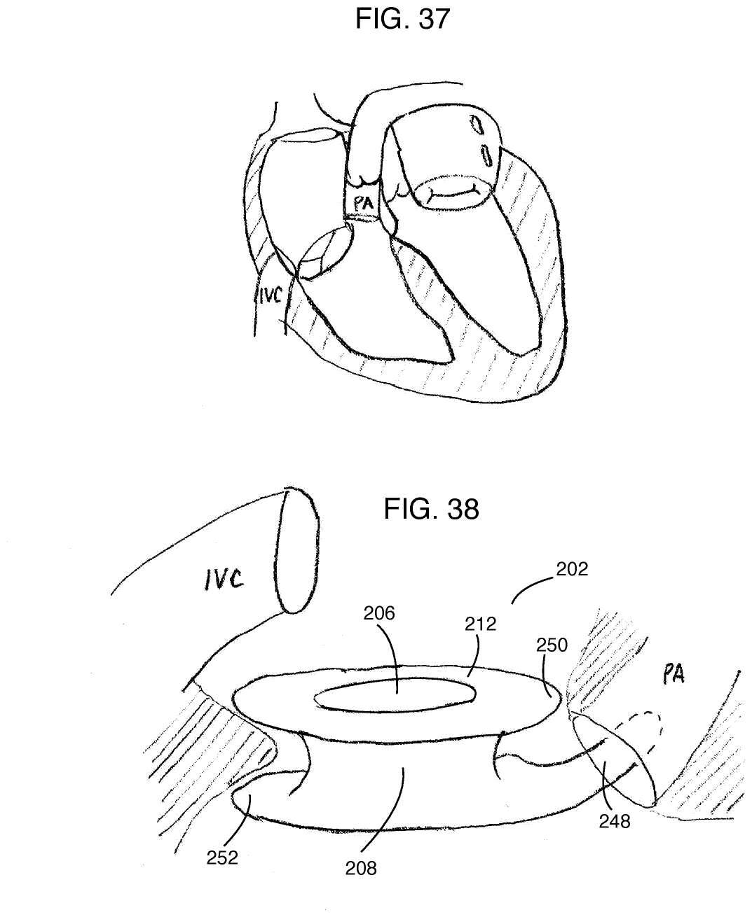

FIG. 37 is an illustration of a side cut-away view of the heart.

FIG. 38 is an illustration of a side-delivered valve prosthesis 202 deployed within the native tricuspid annulus with the inferior vena cava and the pulmonary artery. FIG. 38 shows central channel 206 within collar 212. Upper anchor portion 250 is shown above lower RVOT tab 248. Valve body 208 is shown extending transannularly having subannular RVOT anchor tab 248 extending distally and subannular proximal tab 252 extending proximally.

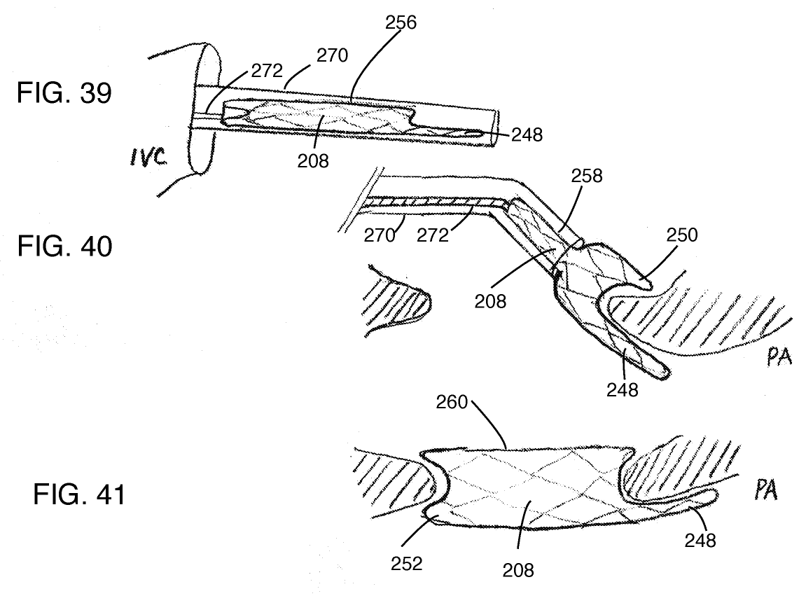

FIG. 39 is an illustration of a delivery catheter 270 extending out from the inferior vena cava from a femoral vein access with a (height) compressed 256 orthogonally (side) deliverable valve 202 disposed within the lumen of the catheter 270 and having deployment tool/rod 272 attached to the near/proximal side of sidewall 208 of the compressed valve 256 for advancing the valve out of the catheter 270. RVOT subannular anchor tab 248 is shown leading the valve 202 along a horizontal axis down the delivery catheter 270.

FIG. 40 is an illustration of a delivery catheter 270 extending out from the inferior vena cava from a femoral vein access with a partially released partially height compressed 258 orthogonally (side) deliverable valve being seated against the distal side of the native tricuspid annulus with a curved (concave) valve perimeter wall (body portion 208), distal side, catching the native annulus within the channel formed by the concave perimeter wall 208 between the distal-side upper anchor portion 250 and the subannular anchor tab 248, with part of the valve still compressed and disposed within the lumen of the catheter and having deployment rod 272 attached to the near/proximal side of the compressed valve for advancing the valve 258 out of the catheter 270. Importantly, the ability to provide a gradual transition from native blood flow to prosthetic blood flow is a critical aspect, by incrementally expelling the compressed valve to commence partial flow through the partially expelled valve, culminating in a transition to full blood flow through the prosthetic valve. This smooth transition avoids a damaging and catastrophic “dry pump” reaction of the heart called a sphincter effect.

FIG. 41 is an illustration of a fully deployed prosthetic valve 260 seated within the native tricuspid annulus. Valve 260 is shown with proximal tab 252 engaging the proximal annular tissue, RVOT tab 248 is shown providing a subannular anchor.

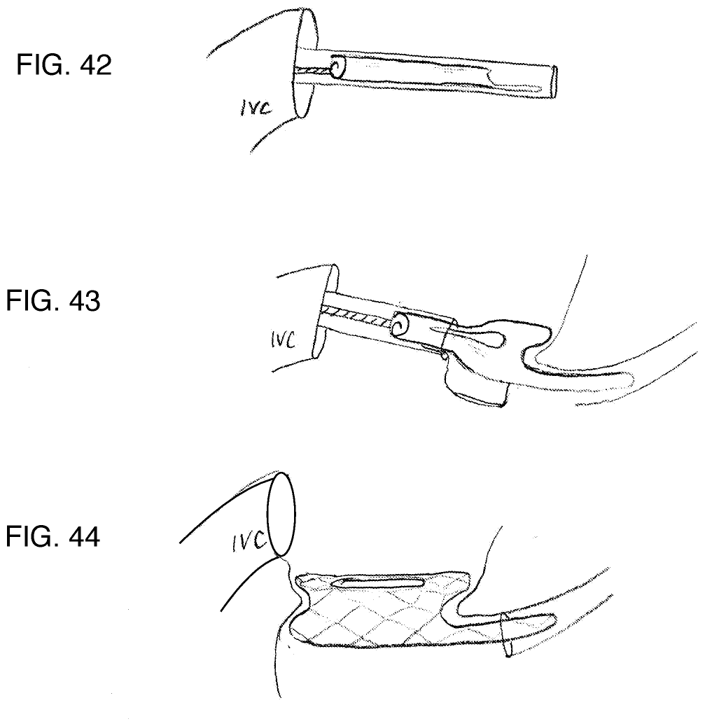

FIG. 42 is an illustration of a delivery catheter extending out from the inferior vena cava from a femoral vein access with a (rolled) compressed orthogonally (side) deliverable valve disposed within the lumen of the catheter and having deployment rod attached to the near/proximal side of the compressed valve for advancing the valve out of the catheter.

FIG. 43 is an illustration of a delivery catheter extending out from the inferior vena cava from a femoral vein access with a partially released partially roll-compressed orthogonally (side) deliverable valve being seated against the distal side of the native tricuspid annulus with a curved (concave) valve perimeter wall (body portion), distal side, catching the native annulus within the channel formed by the concave perimeter wall, with part of the still compressed valve disposed within the lumen of the catheter and having deployment rod attached to the near/proximal side of the compressed valve for advancing the valve out of the catheter. Importantly, the ability to provide a gradual transition from native blood flow to prosthetic blood flow is a critical aspect, by incrementally expelling the compressed valve to commence partial flow through the partially expelled valve, culminating in a transition to full blood flow through the prosthetic valve. This smooth transition avoids a damaging and catastrophic “dry pump” reaction of the heart called a sphincter effect.

FIG. 44 is an illustration of a fully deployed prosthetic valve seated within the native tricuspid annulus.

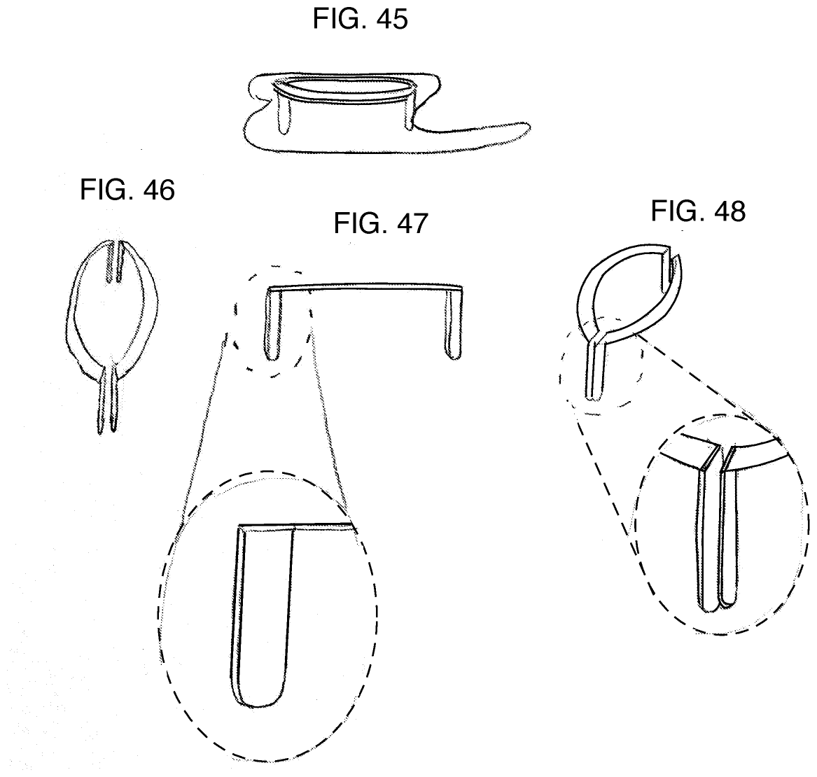

FIG. 45 is an illustration of a side view of a leaflet frame viewed against an outline of a side-delivered prosthetic valve.

FIG. 46 is an illustration of a top, length-wise view down the horizontal-axis (proximal to distal) of a leaflet frame with band or paddle struts in a narrow aspect and annular ring in a wider, flattened aspect.

FIG. 47 is an illustration of a side view of a leaflet frame with a detail zoom of a strut in a band or paddle configuration, with wide axis showing a direction of rigid strut strength and narrow axis showing a direction of flexible strut fold-ability or roll-ability.

FIG. 48 is an illustration of a top-perspective length-wise view down the horizontal-axis (proximal to distal) of a leaflet frame with band or paddle struts in a narrow aspect and annular ring in a wider, flattened aspect, showing paired, parallel band or paddle struts.

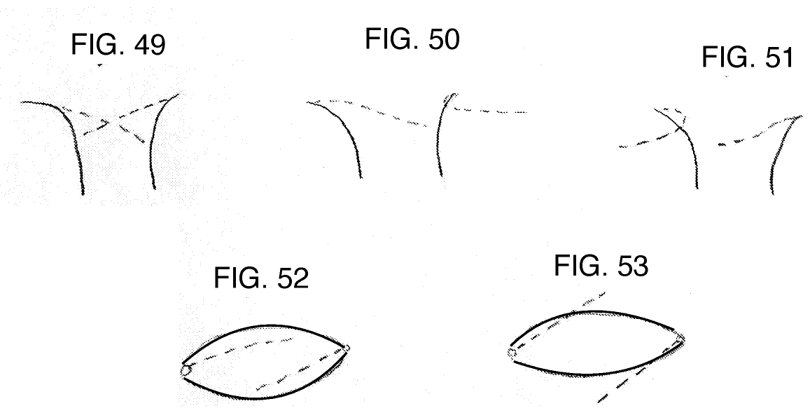

FIG. 49 is an illustration of a side view of one preferred embodiment of a criss-crossed folding direction for struts in a compressed configuration.

FIG. 50 is an illustration of a side view of one preferred embodiment of a distal parallel folding direction for struts in a compressed configuration.

FIG. 51 is an illustration of a side view of one preferred embodiment of a proximal parallel folding direction for struts in a compressed configuration.

FIG. 52 is an illustration of a side view of one preferred embodiment of an internal facing in-plane folding direction for struts in a compressed configuration.

FIG. 53 is an illustration of a side view of one preferred embodiment of an internal facing out-of-plane folding direction for struts in a compressed configuration.

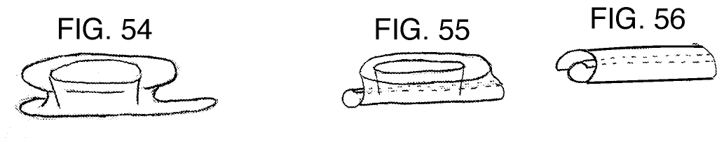

FIG. 54 is an illustration of a side view a leaflet frame as viewed against an outline of a uncompressed pre-rolled (for roll-compression vs. height compression) orthogonal prosthetic valve.

FIG. 55 is an illustration of a side view a leaflet frame as viewed against an outline of a partially compressed mid-roll (for roll-compression vs. height compression) orthogonal prosthetic valve.

FIG. 56 is an illustration of a side view a leaflet frame as viewed against an outline of a fully compressed completely rolled (for roll-compression vs. height compression) orthogonal prosthetic valve.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

Having described embodiments for the invention herein, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

1. A compressible bileaflet frame as a flow control component for a side delivered transcatheter prosthetic valve, comprising:
an annular ring having a distal strut and a proximal strut opposite the distal strut, and
a first leaflet and a second leaflet disposed within the annular ring forming a reciprocating flow control component, each leaflet comprised of pericardium or a biocompatible material,
wherein the annular ring comprises a first semi-ellipsoidal frame member and a second semi-ellipsoidal frame member, each semi-ellipsoidal frame having a distal end and a proximal end, wherein the distal end of the first semi-ellipsoidal frame member and the second semi-ellipsoidal are adjacent, and wherein the proximal end of the first semi-ellipsoidal frame member and the second semi-ellipsoidal are adjacent,
wherein the distal strut is comprised of a first distal post attached length-wise to a second distal post, and the proximal strut is comprised of a first proximal post attached length-wise to a second proximal post, wherein the distal strut and the proximal strut are oriented in an uncompressed configuration at an angle of between 45-135 degrees to a plane of the annular ring,
wherein the first leaflet is attached along a upper portion to the first semi-ellipsoidal frame member, and the first leaflet is attached at a distal portion to the first distal post and at a proximal portion to the first proximal post,
wherein the second leaflet is attached along a upper portion to the second semi-ellipsoidal frame member, and the second leaflet is attached at a distal portion to the second distal post and at a proximal portion to the second proximal post, and
wherein the annular ring has a diameter of about 25-80 mm and the distal and proximal struts have a length of about 5-60 mm.

2. The compressible bileaflet frame of claim 1, wherein the distal and proximal struts are oriented in an uncompressed configuration at an angle of about 90 degrees to the plane of the annular ring.

3. The compressible bileaflet frame of claim 1, wherein said compressible bileaflet frame is comprised of a braided, wire, or laser-cut wire frame.

4. The compressible bileaflet frame of claim 1, wherein the annular ring, distal strut and proximal strut are a unitary construction.

5. The compressible bileaflet frame of claim 1, wherein the first semi-ellipsoidal frame member and second semi-ellipsoidal frame member form a circle, wherein a bisecting axis of each of the first semi-ellipsoidal frame member and second semi-ellipsoidal frame member is equal to a diameter of of each of the first semi-ellipsoidal frame member and a second semi-ellipsoidal frame member.

6. The compressible bileaflet frame of claim 1, wherein the first semi-ellipsoidal frame member and second semi-ellipsoidal frame member are formed from a flat band of superelastic shape-memory alloy, or from a round-wire form of a superelastic shape-memory alloy, or a composite of a flat band and a round-wire form of a superelastic shape-memory alloy.

7. The compressible bileaflet frame of claim 1, wherein the distal strut and proximal strut are formed from a flat band of superelastic shape-memory alloy.

8. The compressible bileaflet frame of claim 1, wherein the first leaflet and the second leaflet coapt along a symmetric long-axis (distal-proximal), or coapt along a symmetric short-axis (septal-anterior), or coapt along an asymmetric axis.

9. The compressible bileaflet frame of claim 1, wherein at least one of the first or second leaflets is supported with one or more longitudinal supports integrated into or mounted upon the pericardium or biocompatible material, the one or more longitudinal supports selected from rigid or semi-rigid posts, rigid or semi-rigid ribs, rigid or semi-rigid battons, rigid or semi-rigid panels, and combinations thereof.

10. A side delivered transcatheter prosthetic heart valve, comprising:
a self-expanding annular support frame, said annular support frame having a central channel and an outer perimeter wall circumscribing a central vertical axis in an expanded configuration,
said perimeter wall having a front wall portion and a back wall portion, the front wall portion and the back wall portion connected along a proximal side to a proximal fold area, and the front wall portion and the back wall portion connected along a distal side to a distal fold area,
the front wall portion having a front upper collar portion and a front lower body portion, the back wall portion having a back upper collar portion and a back lower body portion,
a flow control component mounted within the annular support frame and configured to permit blood flow in a first direction through an inflow end of the valve and block blood flow in a second direction, opposite the first direction, through an outflow end of the valve, wherein the flow control component is a compressible bileaflet frame comprising (i) an annular ring having a distal strut and a proximal strut opposite the distal strut, and (ii) a first leaflet and a second leaflet disposed within the annular ring, each leaflet comprised of pericardium or a biocompatible material, wherein the annular ring comprises a first semi-ellipsoidal frame member and a second semi-ellipsoidal frame member, each semi-ellipsoidal frame having a distal end and a proximal end, wherein the distal end of the first semi-ellipsoidal frame member and the second semi-ellipsoidal are adjacent, and wherein the proximal end of the first semi-ellipsoidal frame member and the second semi-ellipsoidal are adjacent, wherein the distal strut is comprised of a first distal post attached length-wise to a second distal post, and the proximal strut is comprised of a first proximal post attached length-wise to a second proximal post, wherein the distal strut and the proximal strut are oriented in an uncompressed configuration at an angle of between 45-135 degrees to a plane of the annular ring, wherein the first leaflet is attached along a upper portion to the first semi-ellipsoidal frame member, and the first leaflet is attached at a distal portion to the first distal post and at a proximal portion to the first proximal post, wherein the second leaflet is attached along a upper portion to the second semi-ellipsoidal frame member, and the second leaflet is attached at a distal portion to the second distal post and at a proximal portion to the second proximal post, and
wherein the annular ring has a diameter of about 25-80 mm and the distal and proximal struts have a length of about 5-60 mm.
wherein the valve is compressible to a compressed configuration for introduction into the body using a delivery catheter for implanting at a desired location in the body, said compressed configuration is oriented along a horizontal axis at an intersecting angle of between 45-135 degrees to the central vertical axis, and expandable to an expanded configuration having a horizontal axis at an intersecting angle of between 45-135 degrees to the central vertical axis,
wherein the horizontal axis of the compressed configuration of the valve is substantially parallel to a length-wise cylindrical axis of the delivery catheter,
wherein the valve has a height of about 5-60 mm and a diameter of about 25-80 mm.

11. The valve of claim 10, wherein the annular support frame is comprised of a plurality of compressible wire cells having a orientation and cell geometry substantially orthogonal to the central vertical axis to minimize wire cell strain when the annular support frame is configured in a vertical compressed configuration, a rolled compressed configuration, or a folded compressed configuration.

12. The valve of claim 10, wherein the front lower body portion and the back lower body portion in an expanded configuration form a shape selected from a funnel, cylinder, flat cone, or circular hyperboloid.

13. The valve of claim 10, wherein said annular support frame is comprised of a braided, wire, or laser-cut wire frame, and said annular support frame is covered with a biocompatible material.

14. The valve of claim 10, wherein the annular support frame has a side profile of a flat cone shape having a diameter R of 40-80 mm, a diameter r of 20-60 mm, and a height of 5-60 mm.

15. The valve of claim 10, wherein the annular support frame has an inner surface and an outer surface, said inner surface and said outer surface covered with a biocompatible material selected from the following consisting of: the inner surface covered with pericardial tissue, the outer surface covered with a woven synthetic polyester material, and both the inner surface covered with pericardial tissue and the outer surface covered with a woven synthetic polyester material.

16. The valve of claim 10, wherein the annular support frame has a side profile of an hourglass shape having a top diameter R1 of 40-80 mm, a bottom diameter R2 of 50-70 mm, an internal diameter r of 20-60 mm, and a height of 5-60 mm.

17. The valve of claim 10, wherein the valve in an expanded configuration has a central vertical axis that is substantially parallel to the first direction.

18. The valve of claim 10, wherein the flow control component has an internal diameter of 20-60 mm and a height of 10-40 mm, and a plurality of leaflets of pericardial material joined to form a rounded cylinder at an inflow end and having a flat closable aperture at an outflow end.

19. The valve of claim 10, wherein the flow control component is supported with one or more longitudinal supports integrated into or mounted upon the flow control component, the one or more longitudinal supports selected from rigid or semi-rigid posts, rigid or semi-rigid ribs, rigid or semi-rigid battons, rigid or semi-rigid panels, and combinations thereof.

20. The valve of claim 10, comprising a tension arm extending from a distal side of the annular support frame as an RVOT tab, the tension arm comprised of wire loop or wire frame, integrated frame section, or stent, extending from about 10-40 mm away from the annular support frame.

21. The valve of claim 10, comprising (i) an upper tension arm attached to a distal upper edge of the annular support frame, the upper tension arm comprised of wire loop or wire frame extending from about 2-20 mm away from the annular support frame, and (ii) a lower tension arm as an RVOT tab extending from a distal side of the annular support frame, the lower tension arm comprised of wire loop or wire frame, integrated frame section, or stent, extending from about 10-40 mm away from the annular support frame.

22. The valve of claim 10, comprising at least one tissue anchor connected to the annular support frame for engaging native tissue.

23. The valve of claim 10, wherein the front wall portion is a first flat panel and the back wall portion is a second flat panel, and wherein the proximal fold area and the distal fold area each comprise a sewn seam, a fabric panel, or a rigid hinge.

24. The valve of claim 10, wherein the proximal fold area and the distal fold area, each comprise a flexible fabric span without any wire cells.

25. The valve of claim 10, wherein the annular support frame is comprised of compressible wire cells selected from the group consisting of braided-wire cells, laser-cut wire cells, photolithography produced wire cells, 3D printed wire cells, wire cells formed from intermittently connected single strand wires in a wave shape, a zig-zag shape, or spiral shape, and combinations thereof.

26. A method for compressing the implantable prosthetic heart valve of claim 10 for length-wise orthogonal release of the valve from a delivery catheter, comprising the steps:
flattening, rolling or folding the implantable prosthetic heart valve of claim 10 into a compressed configuration wherein the long-axis of the compressed configuration of the valve is substantially parallel to a length-wise cylindrical axis of the delivery catheter,
wherein the implantable prosthetic heart valve comprises an annular support frame having a flow control component mounted within the annular support frame and configured to permit blood flow in a first direction through an inflow end of the valve and block blood flow in a second direction, opposite the first direction, through an outflow end of the valve,
wherein the valve has a height of about 5-60 mm and a diameter of about 25-80 mm
wherein the implantable prosthetic heart valve is rolled or folded into a compressed configuration using a step selected from the group consisting of:
(i) unilaterally rolling into a compressed configuration from one side of the annular support frame;
(ii) bilaterally rolling into a compressed configuration from two opposing sides of the annular support frame;
(iii) flattening the annular support frame into two parallel panels that are substantially parallel to the long-axis, and then rolling the flattened annular support frame into a compressed configuration; and
(iv) flattening the annular support frame along a vertical axis to reduce a vertical dimension of the valve from top to bottom.

27. The method of claim 26, wherein the implantable prosthetic heart valve is rolled or folded into a compressed configuration using a step selected from the group consisting of:
(i) unilaterally rolling into a compressed configuration from one side of the annular support frame;
(ii) bilaterally rolling into a compressed configuration from two opposing sides of the annular support frame;
(iii) flattening the annular support frame into two parallel panels that are substantially parallel to the long-axis, and then rolling the flattened annular support frame into a compressed configuration; and
(iv) flattening the annular support frame along a vertical axis to reduce a vertical dimension of the valve from top to bottom.

28. A method for orthogonal delivery of the implantable prosthetic heart valve of claim 10 to a desired location in the body, the method comprising the steps:
advancing a delivery catheter to the desired location in the body and delivering the expandable prosthetic heart valve of claim 10 to the desired location in the body by releasing the valve from the delivery catheter
wherein releasing the valve from the delivery catheter is selected from the steps consisting of: (i) pulling the valve out of the delivery catheter using a rigid elongated pushing rod/draw wire that is releasably connected to the distal side of the valve, wherein advancing the pushing rod away from the delivery catheter pulls the compressed valve out of the delivery catheter, or (ii) pushing the valve out of the delivery catheter using a rigid elongated pushing rod that is releasably connected to the proximal side of the valve, wherein advancing the pushing rod out of from the delivery catheter pushes the compressed valve out of the delivery catheter,
positioning a lower tension arm of the heart valve prosthesis into the right ventricular outflow tract of the right ventricle, and positioning an upper tension arm into a supra-annular position, and the upper tension arm providing a supra-annular downward force in the direction of the ventricle and lower tension arm providing a sub-annular upward force in the direction of the atrium.

29. The method of claim 28, wherein releasing the valve from the delivery catheter is selected from the steps consisting of: (i) pulling the valve out of the delivery catheter using a rigid elongated pushing rod/draw wire that is releasably connected to the distal side of the valve, wherein advancing the pushing rod away from the delivery catheter pulls the compressed valve out of the delivery catheter, or (ii) pushing the valve out of the delivery catheter using a rigid elongated pushing rod that is releasably connected to the proximal side of the valve, wherein advancing the pushing rod out of from the delivery catheter pushes the compressed valve out of the delivery catheter.

30. The method of claim 28, comprising the additional step of anchoring one or more tissue anchors attached to the valve into native tissue.

31. The method of claim 28, comprising the additional step of positioning a tension arm of the heart valve prosthesis into the right ventricular outflow tract of the right ventricle.

32. The method of claim 28, comprising the additional steps of positioning a lower tension arm of the heart valve prosthesis into the right ventricular outflow tract of the right ventricle, and positioning an upper tension arm into a supra-annular position, and the upper tension arm providing a supra-annular downward force in the direction of the ventricle and lower tension arm providing a sub-annular upward force in the direction of the atrium.

33. The method of claim 28, comprising the the additional step of rotating the heart valve prosthesis using a steerable catheter along an axis parallel to the plane of the valve annulus, wherein an upper tension arm mounted on the valve is conformationally pressure locked against supra-annular tissue, and wherein a lower tension arm mounted on the valve is conformationally pressure locked against sub-annular tissue.
